(19) United States
(12) Patent Application Publication
(10) Pub. No.: US 2006/0030600 A1
(43) Pub. Date: Feb. 9, 2006

(54) Dual NK1/NK3 receptor antagonists for the treatment of schizophrenia

(76) Inventor: Patrick Schnider, Bottmingen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/189,404

(22) Filed: Jul. 26, 2005

(30) Foreign Application Priority Data
Aug. 6, 2004 (EP) 04103794.6

(51) Int. Cl. A61K 31/4412

(52) U.S. Cl. 514/346

(57) ABSTRACT

The present invention relates to a method of treating schizophrenia which comprises administering a therapeutically effective amount of a compound of formula I 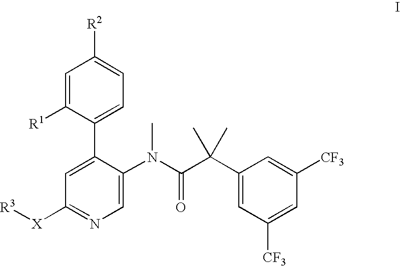 wherein
R¹, R², and R³ are as defined in the specification or to pharmaceutically active acid-addition salts thereof.

### BACKGROUND OF THE INVENTION

Schizophrenia is one of the major neuropsychiatric disorders, characterized by severe and chronic mental impairment. This devastating disease affects about 1% of the world's population. Symptoms begin in early adulthood and are followed by a period of interpersonal and social dysfunction. Schizophrenia manifests as auditory and visual hallucinations, paranoia, delusions (positive symptoms), blunted affect, depression, anhedonia, poverty of speech, memory and attention deficits, as well as social withdrawal (negative symptoms).

For decades scientists and clinicians have made efforts with the aim of discovering an ideal agent for the pharmacological treatment of schizophrenia. However, the complexity of the disorders, due to a wide array of symptoms, has hampered those efforts. There are no specific focal characteristics for the diagnosis of schizophrenia and no single symptom is consistently present in all patients. Consequently, the diagnosis of schizophrenia as a single disorder or as a variety of different disorders has been discussed but not yet resolved. The major difficulty in the development of a new drug for schizophrenia is the lack of knowledge about the cause and nature of this disease. Some neurochemical hypotheses have been proposed on the basis of pharmacological studies to rationalize the development of a corresponding therapy: the dopamine, the serotonin and the glutamate hypotheses. But taking into account the complexity of schizophrenia, an appropriate multireceptor affinity profile might be required for efficacy against positive and negative signs and symptoms. Furthermore, an ideal drug against schizophrenia would preferably have a low dosage allowing once-per-day dosage, due to the low adherence of schizophrenic patients.

In recent years clinical studies with selective NK1 and NK2 receptor antagonists appeared in the literature showing results for the treatment of emesis, depression, anxiety, pain and migraine (NK1) and asthma (NK2 and NK1). The most exciting data were produced in the treatment of chemotherapy-induced emesis, nausea and depression with NK1 and in asthma with NK2-receptor antagonists. In contrast, no clinical data on NK3 receptor antagonists have appeared in the literature until 2000. Osanetant (SR 142,801) from Sanofi-Synthelabo was the first identified potent and selective non-peptide antagonist described for the NK3 tachykinin receptor for the potential treatment of schizophrenia, which was reported in the literature (Current Opinion in Investigational Drugs, 2001,2(7), 950-956 and Psychiatric Disorders Study 4, Schizophrenia, June 2003, Decision Recources, Inc., Waltham, Mass.). The proposed drug SR 142,801 has been shown in a phase II trial as active on positive symptoms of schizophrenia, such as altered behaviour, delusion, hallucinations, extreme emotions, excited motor activity and incoherent speech, but inactive in the treatment of negative symptoms, which are depression, anhedonia, social isolation or memory and attention deficits.

The neurokinin-3 receptor antagonists have been described as useful in pain or inflammation, as well as in schizophrenia, Exp. Opinion. Ther. Patents (2000), 10(6), 939-960 and Current Opinion in Investigational Drugs, 2001, 2(7), 950-956 956 and Psychiatric Disorders Study 4, Schizophrenia, June 2003, Decision Recources, Inc., Waltham, Mass.).

In addition, EP 1 192 952 describes a pharmaceutical composition containing a combination of a NK3 receptor antagonist and a CNS penetrant NK1 receptor antagonist for the treatment of depression and anxiety.

### SUMMARY OF THE INVENTION

The invention provides a method for treating schizophrenia by administering a therapeutically effective amount of a compound of formula I 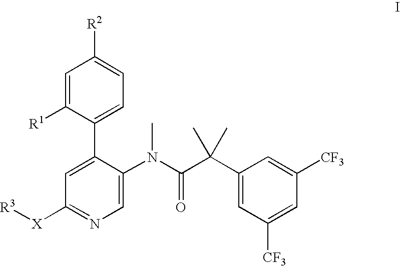
wherein
R¹ is lower alkyl or halogen;
R² is hydrogen or halogen;
R³ is —(CHR′)ₙOH,
   phenyl optionally substituted by —(CHR′)ₙOH,
   a saturated, partially saturated or aromatic 5-or 6-membered heterocyclic ring with one heteroatom or heterogroup, selected from the group consisting of —N(R⁴)—, —N═, 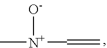
      —S— and —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
or to a pharmaceutically active acid-addition salt thereof.

The compounds of formula I may contain some asymmetric carbon atoms. Accordingly, the present invention includes all stereioisomeric forms of the compounds of formula I, including each of the individual enantiomers and mixtures thereof.

Compounds of formula I and their salts have a high affinity to both the NK1 and the NK3 receptors (dual NK1/NK3 receptor antagonists), useful in the treatment of schizophrenia. In particular, compounds of formula I and pharmaceutically acceptable salts thereof are useful for treating both positive and negative symptoms in schizophrenia.

Some of the compounds of formula I are known compounds, described in EP 1035115, WO 02/08232 or in WO 02/16324. The present invention provides other novel compounds within formula I and pharmaceutically acceptable salts thereof, including each of the individual enantiomers and mixtures thereof.

The present invention further provides methods for the manufacture of compounds of formula and their pharmaceutically acceptable salts. The invention also provides methods for treating illnesses of the kind referred to above by administering a therapeutically effective amount of compounds of formula I.

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, the term “lower alkyl” denotes a straight- or branched-chain alkyl group containing from 1-4 carbon atoms, for example, methyl, ethyl, propyl, isopropyl, n-butyl, i-butyl, t-butyl and the like. The term “alkyl” denotes a straight- or branched-chain alkyl group containing from 1-7 carbon atoms,

The term “halogen” denotes chlorine, iodine, fluorine and bromine.

A “saturated, partially saturated or aromatic 5-or 6 membered heterocyclic ring with one heteroatom or heterogroup” includes, for example pyrrolidin-2-yl, piperidin-4-yl, piperidin-3-yl, pyridine-4-yl, pyridine-3-yl, tetrahydro-pyridine-4-yl, dihydro-thiopyran-4-yl, hexahydro-thiopyran-4-yl and the like.

“Pharmaceutically acceptable” such as pharmaceutically acceptable carrier, excipient, salts, etc., means pharmacologically acceptable, generally safe, substantially non-toxic to the subject to which the particular compound is administered, and neither biologically nor otherwise undesirable.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

“Therapeutically effective amount” means an amount that is effective to prevent, alleviate or ameliorate symptoms of disease or prolong the survival of the subject being treated.

The combination of the antidepressant, mood enhancing properties of NK1 receptor, antagonism and the antipsychotic effects of NK3 receptor antagonism are suitable to treat both positive and negative symptoms in schizophrenia. This advantage may be realized in the administration of an ideal drug against schizophrenia, for example, a compound of formula I or a pharmaceutically acceptable salt thereof.

Thus, the present invention provides a method for treating schizophrenia by administering a therapeutically effective amount of a compound of formula I 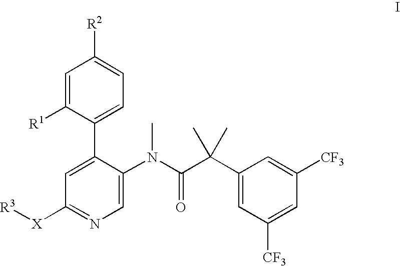
wherein
R¹ is lower alkyl or halogen;
R² is hydrogen or halogen;
R³ is —(CHR′)ₙOH,
   phenyl optionally substituted by —(CHR′)ₙOH,
   a saturated, partially saturated or aromatic 5-or 6-membered heterocyclic ring with one heteroatom or heterogroup, selected from the group consisting of —N(R⁴)—, —N═, 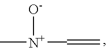
      —S— and —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
or to a pharmaceutically active acid-addition salt thereof. Some of the compounds of formula I are known compounds, described in EP 1035115, WO 02/08232 or in WO 02/16324.

They have been described as active at the NK1 receptor for the treatment of diseases related to this receptor, such as inflammatory conditions including migraine, rheumatoid arthritis, asthma, and inflammatory bowel disease as well as mediation of the emetic reflex and the modulation of central nervous system (CNS) disorders such as Parkinson's disease, anxiety, pain, headache, especially migraine, Alzheimer's disease, multiple sclerosis, attenuation of morphine withdrawal, cardiovascular changes, oedema, such as oedema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases.

The neurokinin-1 receptor antagonists are further useful for the treatment of motion sickness, for treatment induced vomiting or for the treatment of psychoimmunologic or psychosomatic disorders, see Neurosci. Res., 1996, 7, 187-214, Can. J. Phys., 1997, 75, 612-621, Science, 1998, 281, 1640-1645, Auton. Pharmacol., 13, 23-93, 1993, WO 95/16679, WO 95/18124 and WO 95/23798, The New England Journal of Medicine, Vol. 340, No.3 190-195, 1999, U.S. Pat. No. 5,972,938.

The present invention provides a method of using these compounds for the treatment of schizophrenia.

Preferred compounds of formula I for treating schizophrenia are those, wherein X is —O— or —CH₂O—, for example the compounds
2-(3,5-bis-trifluoromethyl-phenyl)-N-[4-(2-chloro-phenyl)-6-(2-hydroxy-ethoxy)-pyridin-3-yl]-N-methyl-isobutyramide,
2-(3,5-bis-trifluoromethyl-phenyl)-N-[4-(2-chloro-phenyl)-6-(2-hydroxy-1-hydroxymethyl-ethoxy)-pyridin-3-yl]-N-methyl-isobutyramide and
(S)-2-(3,5-bis-trifluoromethyl-phenyl)-N-[4-(4-fluoro-2-methyl-phenyl)-6-(pyrrolidin-2-ylmethoxy)-pyridin-3-yl]-N-methyl-isobutyramide.

Further preferred are compounds of formula I, wherein X is —S—, for example 2-(3,5-bis-trifluoromethyl-phenyl)-N-[4-(4-fluoro-2-methyl-phenyl)-6-(2-hydroxy-ethylsulfanyl)-pyridin-3-yl]-N-methyl-isobutyramide.

A further preferred group of compounds are those, wherein X is a bond and R³ is a saturated, partial saturated or aromatic 5-or 6-membered heterocyclic ring with one heteroatom or heterogroup, selected from the group consisting of —N(R⁴)—, —N═, 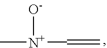
—S— or —S(O)₂, and which rings are optionally substituted by —(CHR′)ₙOH. Compound of this group include the following:
2-(3,5-bis-trifluoromethyl-phenyl)-N-[4-(4-fluoro-2-methyl-phenyl)-[2,3′]bipyridinyl-5-yl]-N-methyl-isobutyramide,
2-(3,5-bis-trifluoromethyl-phenyl)-N-[4-(4-fluoro-2-methyl-phenyl)-1′-oxy-[2,3′]bipyridinyl-5-yl]-N-methyl-isobutyramide,
2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide,
2-(3,5-bis-trifluoromethyl-phenyl)-N-[4-(4-fluoro-2-methyl-phenyl)-5′-hydroxymethyl-[2,3′]bipyridinyl-5-yl]-N-methyl-isobutyramide,
2-(3,5-bis-trifluoromethyl-phenyl)-N-[4-(4-fluoro-2-methyl-phenyl)-2′-hydroxymethyl-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide,
(RS)-2-(3,5-bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide,
(RS)-N-[1′-acetyl-4-(4-fluoro-2-methyl-phenyl)-1′,2′,3′,4′,5′,6′-hexahydro-[2,3′]bipyridinyl-5-yl]-2-(3,5-bis-trifluoromethyl-phenyl)-N-methyl-isobutyramide,
2-(3,5-bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide,
2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide and
2-(3,5-bis-trifluoromethyl-phenyl)-N-[6-(1,1-dioxo-hexahydro-1λ⁶-thiopyran-4-yl)-4-(4-fluoro-2-methyl-phenyl)-pyridin-3-yl]-N-methyl-isobutyramide.

The present invention provides novel compounds of formula I. Examples of such compounds are
2-(3,5-bis-trifluoromethyl-phenyl)-N-[4-(2-chloro-phenyl)-6-(2-hydroxy-ethoxy)-pyridin-3-yl]-N-methyl-isobutyramide,
2-(3,5-bis-trifluoromethyl-phenyl)-N-[4-(2-chloro-phenyl)-6-(2-hydroxy-1-hydroxymethyl-ethoxy)-pyridin-3-yl]-N-methyl-isobutyramide,
(S)-2-(3,5-bis-trifluoromethyl-phenyl)-N-[4-(4-fluoro-2-methyl-phenyl)-6-(pyrrolidin-2-ylmethoxy)-pyridin-3-yl]-N-methyl-isobutyramide.
2-(3,5-bis-trifluoromethyl-phenyl)-N-[4-(4-fluoro-2-methyl-phenyl)-6-(2-hydroxy-ethylsulfanyl)-pyridin-3-yl]-N-methyl-isobutyramide.
2-(3,5-bis-trifluoromethyl-phenyl)-N-[4-(4-fluoro-2-methyl-phenyl)-[2,3′]bipyridinyl-5-yl]-N-methyl-isobutyramide,
2-(3,5-bis-trifluoromethyl-phenyl)-N-[4-(4-fluoro-2-methyl-phenyl)-1′-oxy-[2,3′]bipyridinyl-5-yl]-N-methyl-isobutyramide,
2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide,
2-(3,5-bis-trifluoromethyl-phenyl)-N-[4-(4-fluoro-2-methyl-phenyl)-5′-hydroxymethyl-[2,3′]bipyridinyl-5-yl]-N-methyl-isobutyramide,
2-(3,5-bis-trifluoromethyl-phenyl)-N-[4-(4-fluoro-2-methyl-phenyl)-2′-hydroxymethyl-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide,
(RS)-2-(3,5-bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide,
(RS)-N-[1′-acetyl-4-(4-fluoro-2-methyl-phenyl)-1′,2′,3′,4′,5′,6′-hexahydro-[2,3′]bipyridinyl-5-yl]-2-(3,5-bis-trifluoromethyl-phenyl)-N-methyl-isobutyramide,
2-(3,5-bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide,
2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide and
2-(3,5-bis-trifluoromethyl-phenyl)-N-[6-(1,1-dioxo-hexahydro-1λ⁶-thiopyran-4-yl)-4-(4-fluoro-2-methyl-phenyl)-pyridin-3-yl]-N-methyl-isobutyramide.

Compounds of formula I and their pharmaceutically acceptable salts can be prepared by methods known in the art, described in schemes 1 to 8 and in specific examples 1 to 16 and, for example, by a process described below, which processes comprise reacting a compound of formula 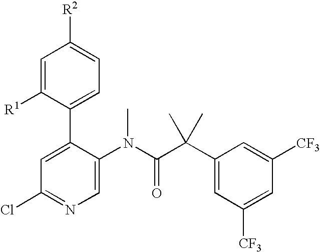
Intermediates 5A-5B with a compound of formula
R³OH
to produce a compound of formula 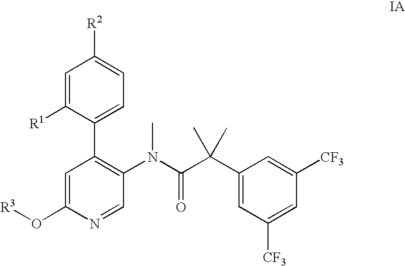
wherein R¹, R² and R³ have the significances given above,
or
reacting a compound of formula 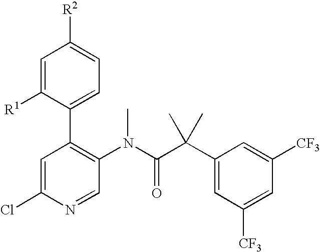
Intermediates 5A-5B with a compound of formula
R³SH
to produce a compound of formula 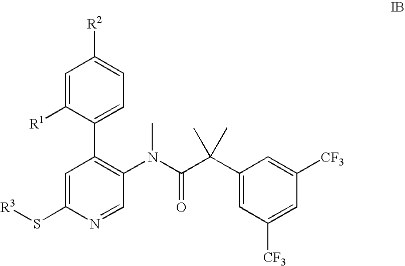
wherein R¹, R² and R³ have the significances given above,
or
reacting a compound of formula 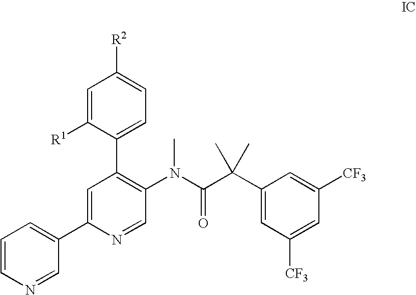
with 3-chloroperbenzoic acid
to produce a compound of formula 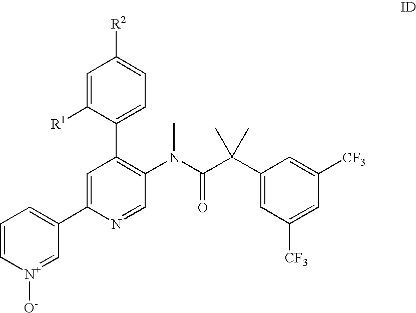
wherein R¹ and R² have the significances given above,
reacting a compound of formula 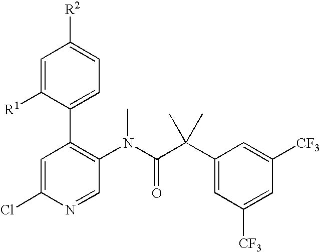
Intermediates 5A-5B with a compound of formula to produce a compound of formula 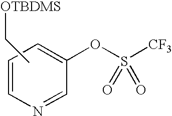 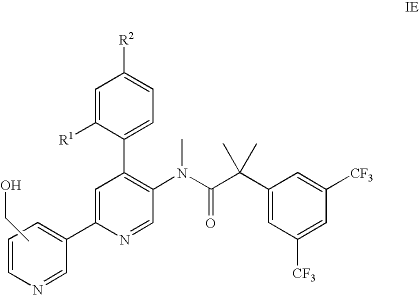
wherein R¹ and R² have the significances given above,
or
reacting a compound of formula 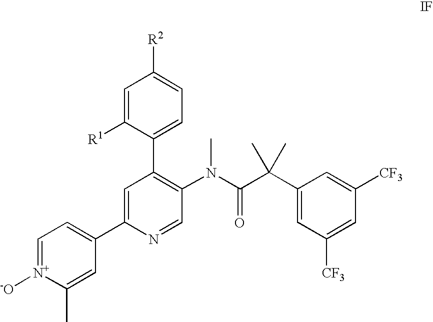
with a compound of formula
(CF₃CO)₂O
to produce a compound of formula 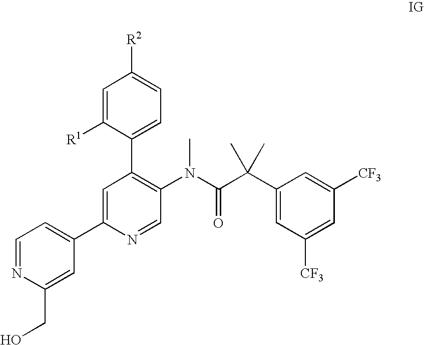
wherein R¹ and R² have the significances given above,
or
reacting a compound of formula 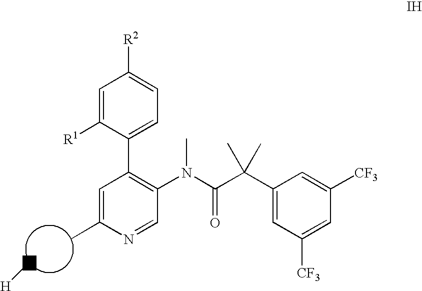
with a compound of formula
CH3SO₂Cl
to produce a compound of formula 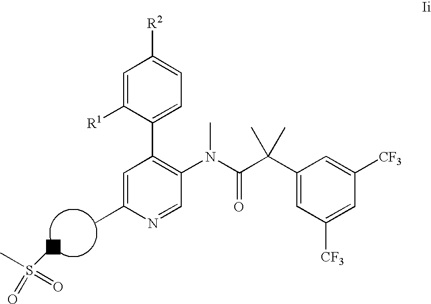
wherein R¹ and R² have the significances given above,
or
reacting a compound of formula 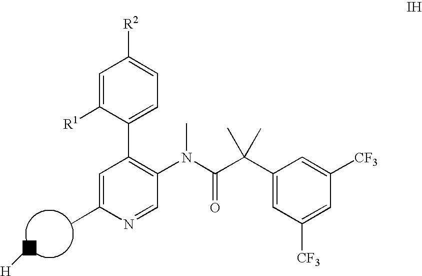
with a compound of formula
(CH₃CO)₂O
to produce a compound of formula 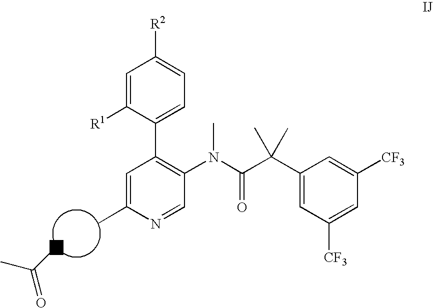
wherein R¹ and R² have the significances given above,
or
reacting a compound of formula 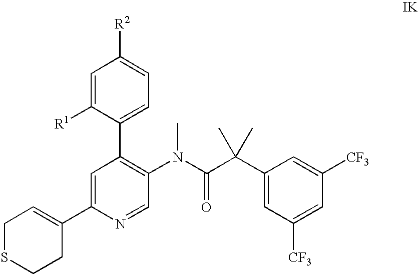
with the compound 3-chloroperbenzoic acid
to produce a compound of formula 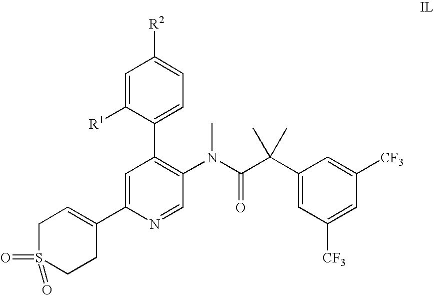
wherein R¹ and R² have the significances given above,
or
hydrogenating a compound of formula 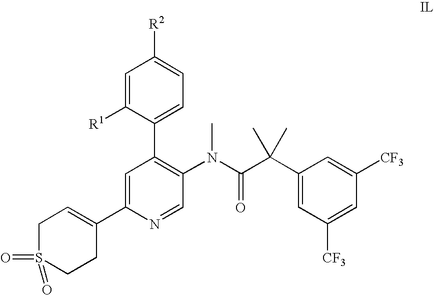
to produce a compound of formula 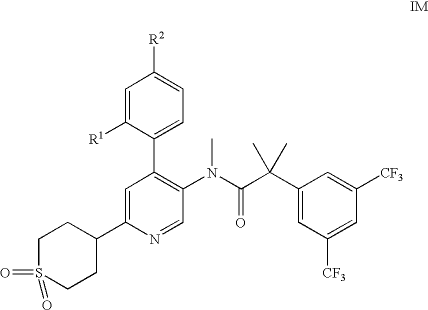
wherein R¹ and R² have the significances given above, and if desired, converting the compound obtained into a pharmaceutically acceptable acid addition salt.

In general, the compounds of formula I may be prepared as follows:

In the schemes the following abbreviations have been used
DMF N,N-dimethylformamide
TFA trifluoroacetic acid
DME ethylene glycol dimethyl ether
KHMDS potassium hexamethyldisilazide
TBDMS tert-butyldimethylsilyl-protecting group
THF tetrahydrofuran
MCPBA 3-chloroperbenzoic acid
dppf 1,1′-bis(diphenylphosphino)ferrocene
RT room temperature 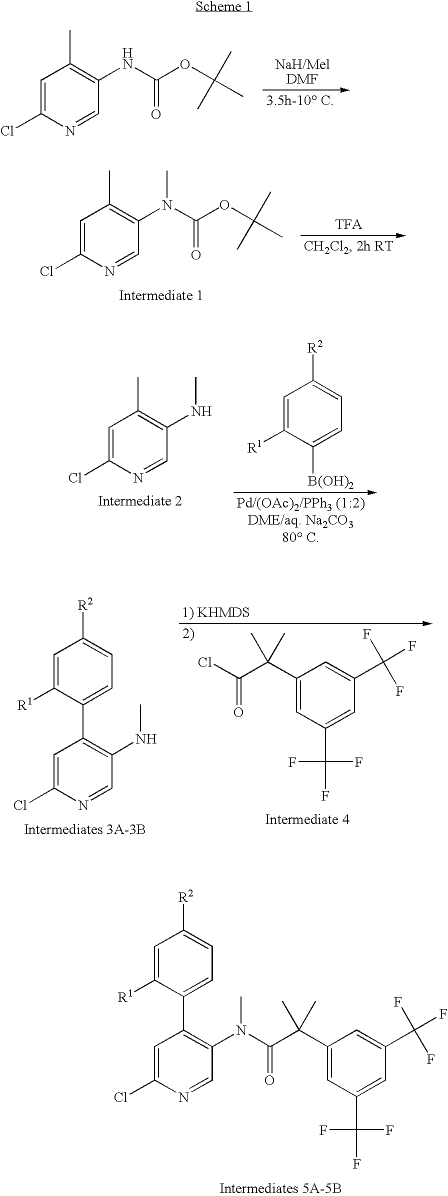
   wherein R¹ has the meaning as described above.

According to scheme 1, the intermediates 5A-5B can be prepared as follows:

Intermediate 1

To a solution of (6-chloro-4-iodo-pyridin-3-yl)-carbamic acid tert-butyl ester in DMF is added sodium hydride at about −10° C. (The preparation of (6-chloro-4-iodo-pyridin-3-yl)-carbamic acid tert-butyl ester has been described in US 2002/0022624 A1, incorporated by reference herein.) The reaction mixture is allowed to warm to room temperature. After about 1 h, the mixture is cooled back and iodomethane is added.

Intermediate 2

To a solution of (6-chloro-4-iodo-pyridin-3-yl)-methyl-carbamic acid tert-butyl ester in dichloromethane is added trifluoroacetic acid at 0° C.

Intermediate 3A

A mixture of (6-chloro-4-iodo-pyridin-3-yl)-methyl-amine, 2-chlorophenylboronic acid, palladium(II) acetate, triphenylphosphine, sodium carbonate solution and 1,2-dimethoxyethane is heated at about 80° C. for 90 min.

Intermediate 3B

The intermediate 3B is obtained according to the procedure described above for the preparation of [6-chloro-4-(2-chloro-phenyl)-pyridin-3-yl)-methyl-amine using 4-fluoro-2-methyl-phenylboronic acid instead of 2-chlorophenylboronic acid.

Intermediate 4

The intermediate 4 is obtained according to the procedure described in WO 02/79134 A1, incorporated by reference herein.

Intermediate 5A

To a solution [6-chloro-4-(2-chloro-phenyl)-pyridin-3-yl]-methyl-amine (intermediate 3A) tetrahydrofuran is added dropwise at about 0° C. a solution of potassium bis(trimethylsilyl)amide in tetrahydrofuran. The reaction mixture is stirred at room temperature for 30 min. After cooling to 0° C. 2-(3,5-bis-trifluoromethyl-phenyl)-2-methyl-propionyl chloride (intermediate 4) is added. The reaction mixture is allowed to warm to room temperature and stirred at room temperature for about 1 h.

Intermediate 5B

The intermediate 5B is obtained according to the procedure described above for the preparation of 2-(3,5-bis-trifluoromethyl-phenyl)-N-[6-chloro-4-(2-chloro-phenyl)-pyridin-3-yl]-N-methyl-isobutyramide (intermediate 5A) using [6-chloro-4-(4-fluoro-2-methyl-phenyl)-pyridin-3-yl]-methyl-amine (intermediate 3B) instead of [6-chloro-4-(2-chloro-phenyl)-pyridin-3-yl]-methyl-amine (intermediate 3A).

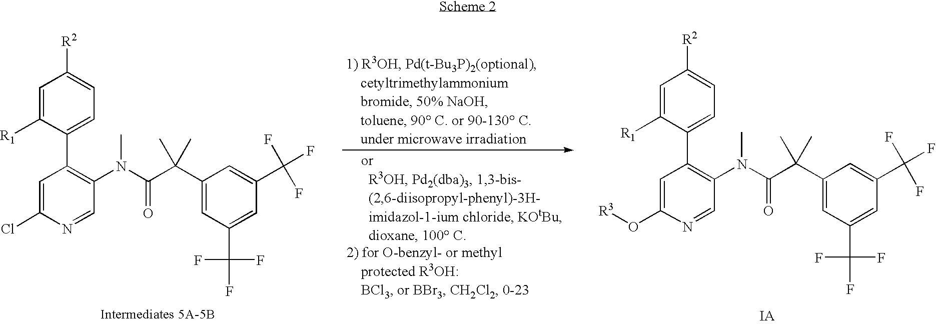

Compounds of formula IA maybe prepared as follows:

A mixture of an intermediate 5A-5B, a corresponding alcohol, such as L-prolinol or 2-(benzyloxy)ethanol or 1,3-dimethoxy-2-propanol, cetyltrimethylammonium bromide, bis(tri-tert-butylphosphine)palladium, NaOH and toluene is degassed by two freeze-thaw cycles. The reaction mixture is heated under argon at about 90° C. for up to 3 days.

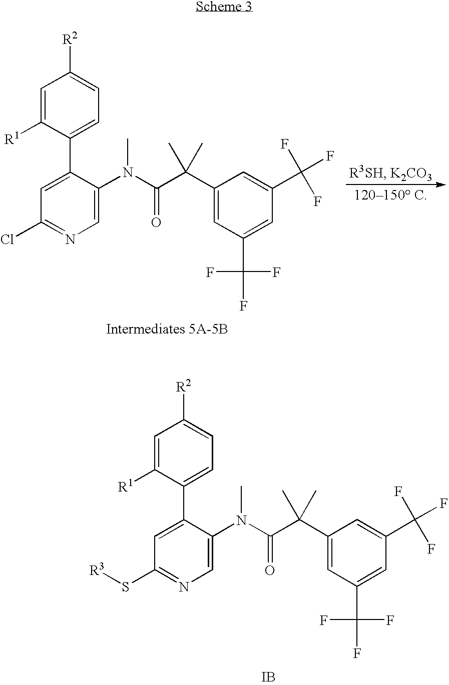

Compounds of formula IB maybe prepared as follows:

A mixture of intermediate 5A-5B, 2-mercapto-alcohol and potassium carbonate is heated under argon at about 140° C. for 3 h.

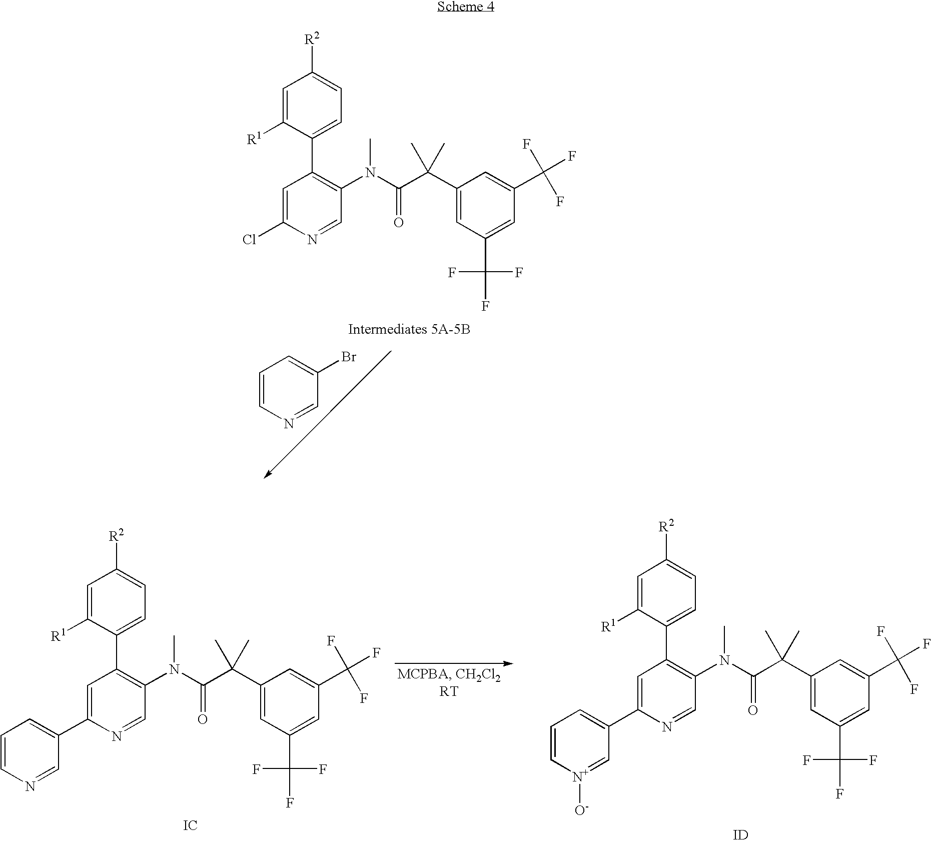

Compounds of formula IC may be prepared as follows:

To a solution of 3-bromopyridine in THF is added a solution of isopropyl magnesium chloride in THF at about −60° C. The resulting solution is kept at −40° C. for about 15 min and is then allowed to warm to room temperature. Then a solution of zinc chloride in THF is added to the suspension. This mixture is stirred for 2 h at room temperature. After addition of a solution of the intermediate 5A-5B and tetrakis(triphenylphosphine)palladium in THF the reaction mixture is heated for about 16 h.

Compound of formula ID may be prepared as follows:
To a solution of a compound of formula IC, for example 2-(3,5-bis-trifluoromethyl-phenyl)-N-[4-(4-fluoro-2-methyl-phenyl)-[2,3′]bipyridinyl-5-yl]-N-methyl-isobutyramide in dichloromethane is added 3-chloroperbenzoic acid at room temperature.

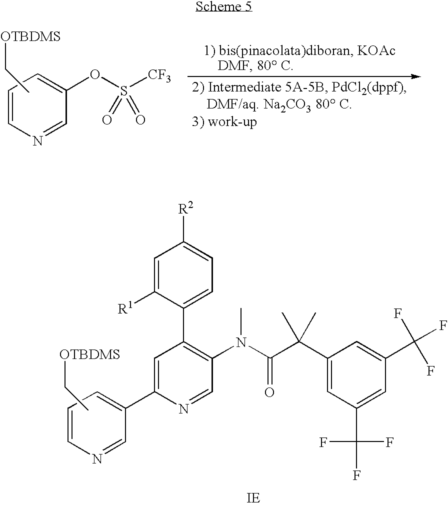

Trifluoro-methanesulfonic acid 5-(tert-butyl-dimethyl-silanyloxymethyl)-pyridin-3-yl ester may be prepared as follows:

To a solution of 5-(tert-butyl-dimethyl-silanyloxymethyl)-pyridin-3-ol (the preparation is described in detail in example 8a to 8d) and triethylamine in dichloromethane is added a solution of trifluoromethanesulfonic anhydride in dichloromethane at 0° C.

A mixture of trifluoro-methanesulfonic acid 5-(tert-butyl-dimethyl-silanyloxymethyl)-pyridin-3-yl ester, bis(pinacolato)diboron and potassium acetate in N,N-dimethylformamide is deoxygenated by three freeze-thaw cycles. The reaction mixture is stirred at about 80° C. over night. After addition of one portion dichloro(1,1′-bis(diphenylphosphino)ferrocene)palladium(II)dichloromethane adduct cooling to room temperature is followed by addition of a solution of sodium carbonate, 2-(3,5-bis-trifluoromethyl-phenyl)-N-[6-chloro-4-(4-fluoro-2-methyl-phenyl)-pyridin-3-yl]-N-methyl-isobutyramide (intermediate 5A-5B) and second portion of dichloro(1,1′-bis(diphenylphosphino)ferrocene)palladium(II) dichloromethane adduct. The reaction mixture is heated at about 80° C. over night and then extracted, washed, concentrated and purified in conventional manner.

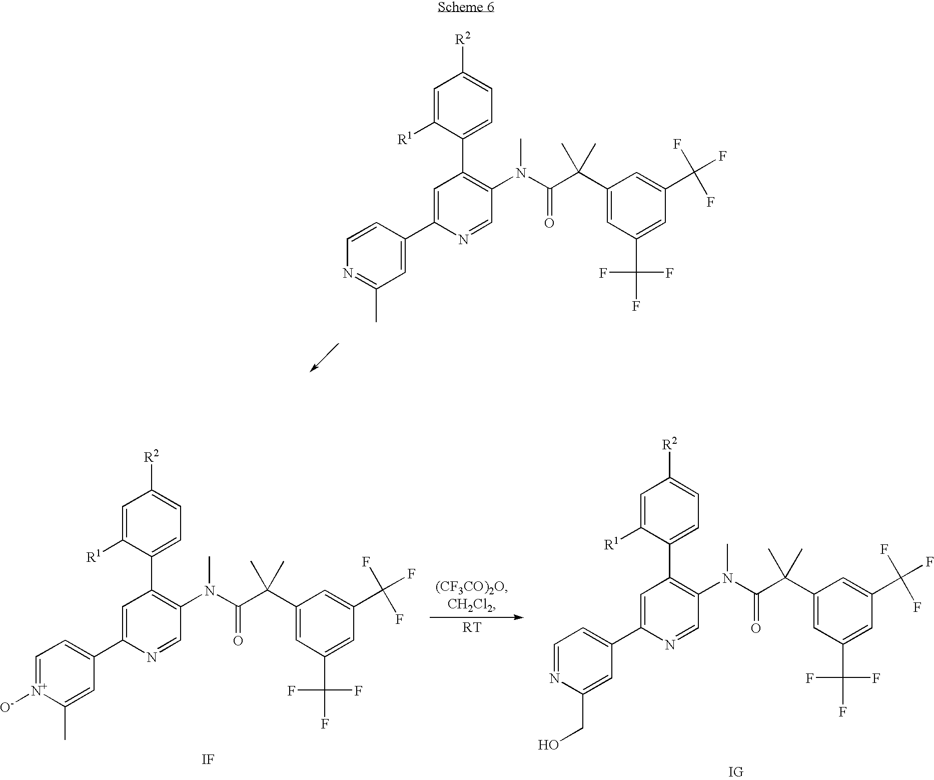

The preparation of the intermediates, for example 2-(3,5-bis-trifluoromethyl-phenyl)-N-[4-(4-fluoro-2-methyl-phenyl)-2′-methyl-[2,4′]bipyridinyl-5-yl]-N-methyl-isobutyramide a compound of formula IF may be carried out in analogy to the description in scheme 4.

The compound of formula IG, for example 2-(3,5-bis-trifluoromethyl-phenyl)-N-[4-(4-fluoro-2-methyl-phenyl)-2′-hydroxymethyl-[2,4′]bipyridinyl-5-yl]-N-methyl-isobutyramide may be prepared as follows:

A solution of 2-(3,5-bis-trifluoromethyl-phenyl)-N-[4-(4-fluoro-2-methyl-phenyl)-2′-methyl-1′-oxy-[2,4′]bipyridinyl-5-yl]-N-methyl-isobutyramide (IF) and trifluoroacetic anhydride is stirred at room temperature over night. Addition of another portion of trifluoroacetic anhydride and stirring for approximately another 20 h may be necessary to drive conversion to completion.

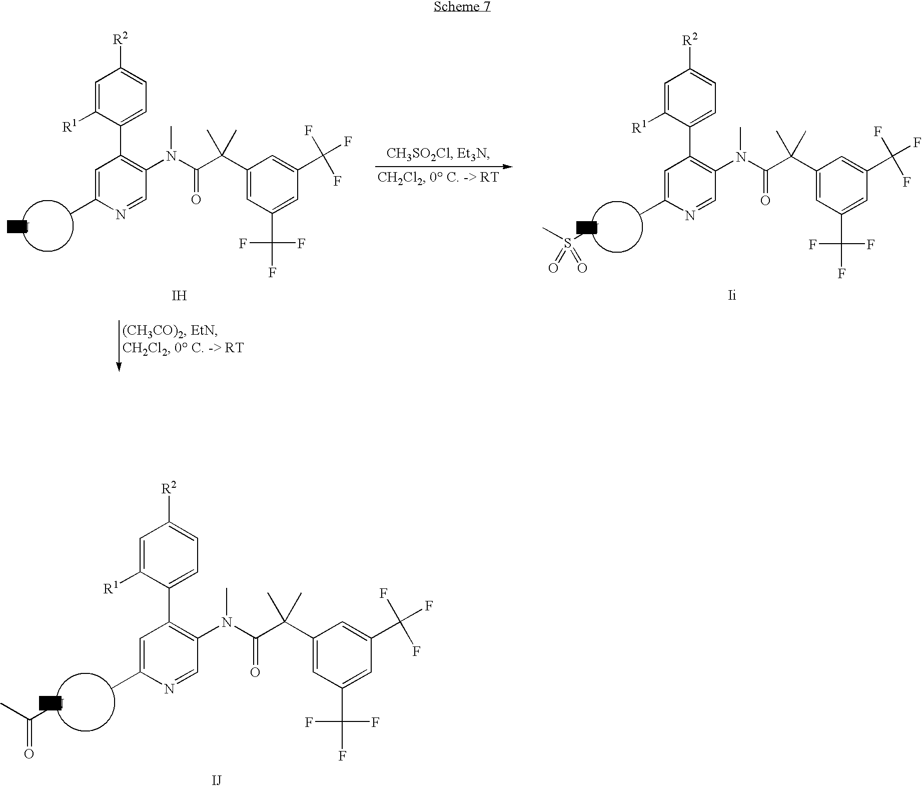

To a solution of this compound of formula IH, for example 2-(3,5-bis-trifluoromethyl-phenyl)-N-[4-(4-fluoro-2-methyl-phenyl)-1′-methanesulfonyl-1′,2′,3′,6′-tetrahydro-[2)4′]bipyridinyl-5-yl]-N-methyl-isobutyramide in dichloromethane is added triethylamine and methanesulfonyl chloride at 0° C.

A compound of formula IJ may be prepared in accordance with the described method for the preparation of compounds of formula Ii, using acetic anhydride instead of methanesulfonyl chloride in the last step.

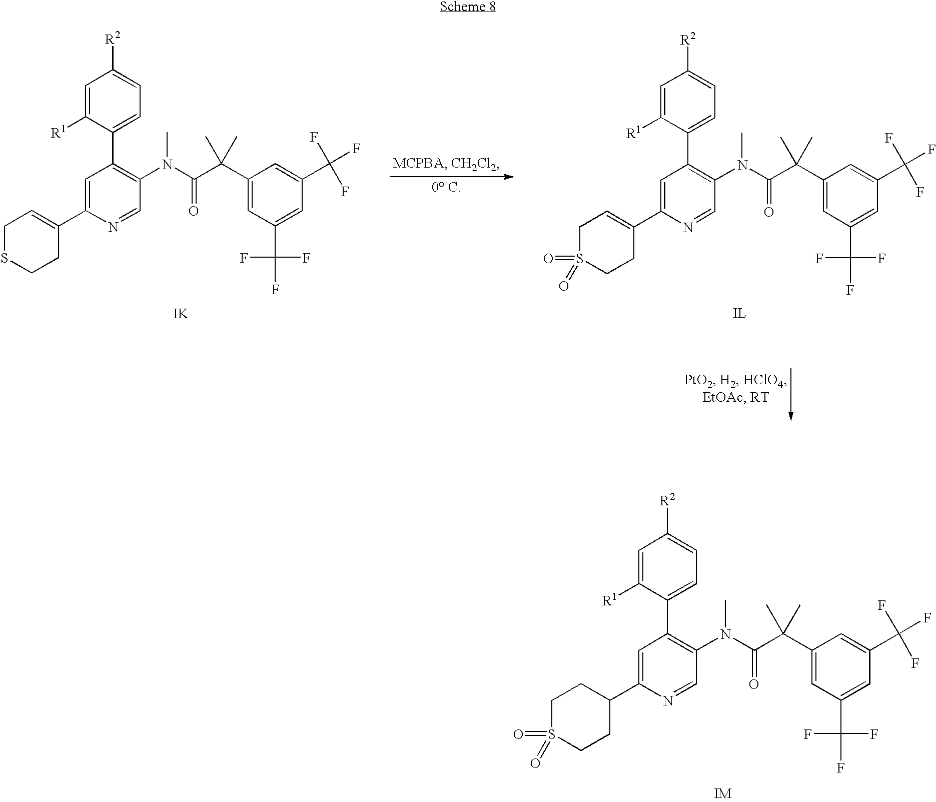

A compound of formula IK is obtained in analogy to the procedure described above for the preparation of 2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide (example 7) using 2-(3,6-dihydro-2H-thiopyran-4-yl)-4,4,5,5-tetramethyl-[1,3,2]dioxaborolane instead of 3-(hydroxymethyl)phenylboronic acid.

To a solution containing a compound of formula IK, for example 2-(3,5-bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide in dichloromethane is added 3-chloroperbenzoic acid at 0° C. After about 3 h the reaction mixture is diluted with a solution of sodium hydroxide, extracted and purified to obtain a compound of formula IL.

Furthermore, a solution of this compound, for example 2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide, and 1 drop of perchloric acid in ethyl acetate is degassed by three freeze-thaw cycles. After addition of platinum(IV)oxide under argon the reaction mixture is stirred under an atmosphere of hydrogen at room temperature for about 6 h to give a compounds of formula IM.

As mentioned earlier, the compounds of formula I and their pharmaceutically acceptable addition salts possess valuable pharmacological properties. These compounds are dual antagonists of the Neurokinin 1 and 3 receptors.

The compounds were investigated in accordance with the tests given hereinafter.

### NK₁

The affinity of test compounds for the NK₁ receptor was evaluated at human NK₁ receptors in CHO cells infected with the human NK₁ receptor (using the Semliki virus expression system) and radiolabelled with [³H]substance P (final concentration 0.6 nM). Binding assays were performed in HEPES buffer (50 mM, pH 7.4) containing BSA (0.04%) leupeptin (16.8 μg/ml), MnCl₂ (3mM) and phosphoramidon (2 μM). Binding assays consisted of 250 μl of membrane suspension (approximately 1.5 μg/well in a 96 well plate), 0.125 μl of buffer of displacing agent and 125 μl of [³H]substance P. Displacement curves were determined with at least seven concentrations of the compound. The assay tubes were incubated for 60 min at room temperature after which time the tube contents were rapidly filtered under vacuum through GF/C filters presoaked for 60 min with PEI (0.3%) with 3×1 ml washes of HEPES buffer (50 mM, pH 7.4). The radioactivity retained on the filters was measured by scintillation counting. All assays were performed in duplicate in at least 2 separate experiments.

### NK₃

Recombinant human NK₃ (hNK₃) receptor affinity was determined in a 96 well plate assay, using [³H]SR142801 (final concentration 0.3 nM) to radiolabel the hNK₃ receptor in the presence of 10 concentrations of competing compound or buffer. Non specific binding was determined using 10 μM SB222200. Assay buffer consisted of Tris-HCl (50 mM, pH 7.4), BSA (0.1%), MnCl₂ (4 mM) and phosphoramidon (1 μM). Membrane preparations of hNK3 receptors (approximately 2.5 μg/well in a 96 well plate) were used to initiate the incubation for 90 min at room temperature. This assay was terminated by rapid filtration under vacuum through GF/C filters, presoaked for 90 min with PEI (0.3%), with 3×0.5 ml washes of ice-cold Tris buffer (50 mM, pH 7.4) containing 0.1% BSA. The radioactivity retained on the filters was measured by scintillation counting. All assays were performed in duplicate in at least two separate experiments.

The activity of the present compounds is described in the table below:

| Example No. | pki NK1 | pki NK3 |
|---|---|---|
| 1 | 9.25 | 7.71 |
| 2 | 8.91 | 8.04 |
| 3 | 9.07 | 7.63 |
| 4 | 8.63 | 8.07 |
| 5 | 8.89 | 7.79 |
| 6 | 9.01 | 7.61 |
| 7 | 8.92 | 8.16 |
| 8 | 8.74 | 8.21 |
| 9 | 8.81 | 7.94 |
| 10 | 9.21 | 8.58 |
| 11 | 8.96 | 8.19 |
| 12 | 9.23 | 7.67 |
| 13 | 8.79 | 7.84 |
| 14 | 8.94 | 7.85 |
| 15 | 9.23 | 8.17 |
| 16 | 8.86 | 7.78 |

### Intermediate 1

### (6-Chloro-4-iodo-pyridin-3-yl)-methyl-carbamic acid tert-butyl ester

To a solution of 1.00 g (2.82 mmol) (6-chloro-4-iodo-pyridin-3-yl)-carbamic acid tert-butyl ester in 10 ml DMF were added 0.12 g (3.1 mmol) sodium hydride (60% in mineral oil) at −10° C. (The preparation of (6-chloro-4-iodo-pyridin-3-yl)-carbamic acid tert-butyl ester has been described in US 2002/0022624 A1.) The reaction mixture was allowed to warm to room temperature. After 1 h, the mixture was cooled back to −10° C., and 0.44 ml (7.1 mmol) iodomethane were added during 5 min. The reaction mixture was allowed to warm to room temperature. After 2.5 at room temperature, the reaction was quenched by addition of 10 ml of a saturated aqueous solution of NaHCO₃ and the mixture was extracted with ethyl acetate. The combined organic layers were washed with brine, dried over Na₂SO₄ and concentrated in vacuo. The crude product was purified by column chromatography (silica gel, hexanes/ethyl acetate=4:1) to give 1.06 g (100%) of the title compound as a colorless oil.

MS m/e (%): 368 (M⁺, 1)

### Intermediate 2

### (6-Chloro-4-iodo-pyridin-3-yl)-methyl-amine

To a solution of 8.65 g (19.6 mmol) (6-chloro-4-iodo-pyridin-3-yl)-methyl-carbamic acid tert-butyl ester in 20 ml dichloromethane were added 20.0 ml (261 mmol) trifluoroacetic acid at 0° C. After stirring for 2 h at room temperature the reaction mixture was concentrated in vacuo. The residue was treated with 50 ml saturated sodium carbonate solution and extracted three times with 75 ml ethyl acetate. The combined organic layers were washed with 50 ml brine, dried over sodium sulfate and concentrated in vacuo to give 6.1 g (87%) of the title compound as a light brown solid.

MS m/e (%): 268 (M⁺, 1)

### Intermediate 3A

### [6-Chloro-4-(2-chloro-phenyl)-pyridin-3-yl]-methyl-amine

A mixture of 6.05 g (19.3 mmol) (6-chloro-4-iodo-pyridin-3-yl)-methyl-amine, 23.6 g (23.6 mmol) 2-chlorophenylboronic acid, 441 mg (1.96 mmol) palladium(II) acetate, 1.03 g (3.93 mmol) triphenylphosphine, 47.1 ml 2 M sodium carbonate solution and 50 ml 1,2-dimethoxyethane was heated at 80° C. for 90 min. The reaction mixture was cooled to room temperature and diluted with 100 ml ethyl acetate. The aqueous layer was separated and extracted with 100 ml ethyl acetate. The combined organic layers were dried over sodium sulfate, concentrated in vacuo and purified by flash chromatography to give 4.1 g (83%) of the title compound as a light brown solid.

MS m/e (%): 253 (M+H⁺, 100)

### Intermediate 3B

### [6-Chloro-4-(4-fluoro-2-methyl-phenyl)-pyridin-3-yl]-methyl-amine

The title compound was obtained as a orange solid in 80% yield after flash chromatography according to the procedure described above for the preparation of [6-chloro-4-(2-chloro-phenyl)-pyridin-3-yl]-methyl-amine using 4-fluoro-2-methyl-phenylboronic acid instead of 2-chlorophenylboronic acid.

MS m/e (%): 251 (M+H⁺, 100)

### Intermediate 4

### 2-(3,5-Bis-trifluoromethyl-phenyl)-2-methyl-propionyl chloride

The title compound is obtained according to the procedure described in WO 0279134 A1.

### Intermediate 5A

### 2-(3,5-Bis-trifluoromethyl-phenyl)-N-[6-chloro-4-(2-chloro-phenyl)-pyridin-3-yl]-N-methyl-isobutyramide

To a solution of 20 g (79 mmol) [6-chloro-4-(2-chloro-phenyl)-pyridin-3-yl]-methyl-amine (intermediate 3A) in 200 ml tetrahydrofuran were added dropwise at 0° C. 113 ml (94.8 mmol) of a 0.91 M solution of potassium bis(trimethylsilyl)amide in tetrahydrofuran. The reaction mixture was stirred at room temperature for 30 min. After cooling to 0° C. 27.7 g (86.9 mmol) 2-(3,5-bis-trifluoromethyl-phenyl)-2-methyl-propionyl chloride (intermediate 4) were added dropwise. The reaction mixture was allowed to warm to room temperature and stirred at room temperature for 1 h. The reaction mixture was treated with 220 ml 1 M sodium hydrogencarbonate solution and extracted with three 200-ml portions of ethyl acetate. The combined organic layers were dried over sodium sulfate and triturated with 150 ml diethylether to give 34.6 g (82%) of the title compound as a white solid.

MS m/e (%): 535 (M+H⁺, 100)

### Intermediate 5B

### 2-(3,5-Bis-trifluoromethyl-phenyl)-N-[6-chloro-4-(4-fluoro-2-methyl-phenyl)-pyridin-3-yl]-N-methyl-isobutyramide

The title compound was obtained as a light yellow foam in 87% yield after flash chromatography according to the procedure described above for the preparation of 2-(3,5-bis-trifluoromethyl-phenyl)-N-[6-chloro-4-(2-chloro-phenyl)-pyridin-3-yl]-N-methyl-isobutyramide (intermediate 5A) using [6-chloro-4-(4-fluoro-2-methyl-phenyl)-pyridin-3-yl]-methyl-amine (intermediate 3B) instead of [6-chloro-4-(2-chloro-phenyl)-pyridin-3-yl]-methyl-amine (intermediate 3A).

MS m/e (%): 533 (M+H⁺, 100)

### EXAMPLE 1

### 2-(3,5-Bis-trifluoromethyl-phenyl)-N-[4-(2-chloro-phenyl)-6-(2-hydroxy-ethoxy)-pyridin-3-yl]-N-methyl-isobutyramide

### N-[6-(2-Benzyloxy-ethoxy)-4-(2-chloro-phenyl)-pyridin-3-yl]-2-(3,5-bis-trifluoromethyl-phenyl)-N-methyl-isobutyramide

A mixture of 0.10 g (0.19 mmol) 2-(3,5-bis-trifluoromethyl-phenyl)-N-[6-chloro-4-(2-chloro-phenyl)-pyridin-3-yl]-N-methyl-isobutyramide, 0.03 ml (0.02 mmol) 2-(benzyloxy)ethanol and 2 ml dioxane was degassed by two freeze-thaw cycles. After addition of 7 mg (0.008 mmol) tris(dibenzylideneacetone)dipalladium(0), 7.0 mg (0.016 mmol) 1,3-bis-(2,6-diisopropyl-phenyl)-3H-imidazol-1-ium chloride and 32 mg (0.29 mmol) potassium tert-butylate the reaction mixture was heated under argon at 100° C. for 2 h. The mixture was cooled to room temperature, followed by addition of 10 mg (0.089 mmol) potassium tert-butylate, 7 mg (0.008 mmol) tris(dibenzylideneacetone)dipalladium(0) and 7.0 mg (0.016 mmol) 1,3-bis-(2,6-diisopropyl-phenyl)-3H-imidazol-1-ium chloride. After heating at 100° C. for another 2 h the reaction mixture was cooled to room temperature, diluted with tert-butyl methyl ether and washed with two portions of water. The combined aqueous layers were extracted with two portions of tert-butyl methyl ether. The combined organic layers were dried over sodium sulphate and concentrated in vacuo. Flash column chromatography gave 60 mg (49%) of the title compound as light yellow, viscous oil.

MS m/e (%): 651 (M+H⁺, 100).

### 2-(3,5-Bis-trifluoromethyl-phenyl)-N-[4-(2-chloro-phenyl)-6-(2-hydroxy-ethoxy)-pyridin-3-yl]-N-methyl-isobutyramide

To a solution of 60 mg (0.092 mmol) N-[6-(2-benzyloxy-ethoxy)-4-(2-chloro-phenyl)-pyridin-3-yl]-2-(3,5-bis-trifluoromethyl-phenyl)-N-methyl-isobutyramide in 2 ml dichloromethane were added 0.13 ml (0.13 mmol) of a 1 M solution of boron trichloride in dichloromethane at room temperature. After consumption of the starting material, 1 ml of a 1 M aqueous solution of hydrochloric acid was added. Dilution with water and 2 ml of a 1 M aqueous solution of sodium hydroxide was followed by extraction with three portions of dichloromethane. The combined organic layers were dried over sodium sulphate and concentrated in vacuo. Flash column chromatography gave 36 mg (70%) of the title compound as an off-white solid.

MS m/e (%): 561 (M+H⁺, 100).

### EXAMPLE 2

### 2-(3,5-Bis-trifluoromethyl-phenyl)-N-[4-(2-chloro-phenyl)-6-(2-hydroxy-1-hydroxymethyl-ethoxy)-pyridin-3-yl]-N-methyl-isobutyramide

### 2-(3,5-Bis-trifluoromethyl-phenyl)-N-[4-(2-chloro-phenyl)-6-(2-methoxy-1-methoxymethyl-ethoxy)-pyridin-3-yl]-N-methyl-isobutyramide

A mixture of 0.15 g (0.28 mmol) 2-(3,5-bis-trifluoromethyl-phenyl)-N-[6-chloro-4-(2-chloro-phenyl)-pyridin-3-yl]-N-methyl-isobutyramide, 0.17 g (1.4 mmol) 1,3-dimethoxy-2-propanol, 5 mg (0.01 mmol) cetyltrimethylammonium bromide, 0.1 ml NaOH 50% and 1 ml toluene was degassed by two freeze-thaw cycles. The reaction mixture was heated under microwave irradiation at 130° C. for 30 min. After cooling to room temperature the mixture was diluted with water and extracted with two portions of tert-butyl methyl ether. The combined organic layers were dried over sodium sulphate and concentrated in vacuo. Flash column chromatography gave 0.11 g (63%) of the title compound as an off-white solid.

MS m/e (%): 619 (M+H⁺, 100).

### 2-(3,5-Bis-trifluoromethyl-phenyl)-N-[4-(2-chloro-phenyl)-6-(2-hydroxy-1-hydroxymethyl-ethoxy)-pyridin-3-yl]-N-methyl-isobutyramide

To a solution of 0.11 g (0.21 mmol) 2-(3,5-bis-trifluoromethyl-phenyl)-N-[4-(2-chloro-phenyl)-6-(2-methoxy-1-methoxymethyl-ethoxy)-pyridin-3-yl]-N-methyl-isobutyramide in 2 ml dichloromethane were added 0.41 ml (0.41 mmol) of a 1 M solution of boron tribromide in dichloromethane at 0° C. The mixture was allowed to warm to room temperature over night. Quenching with a 1 M aqueous solution of hydrochloric acid was followed by extraction with two portions of dichloromethane. The combined organic layers were dried over sodium sulphate and concentrated in vacuo. Flash column chromatography gave 30 mg (25%) of the title compound as an off-white solid.

MS m/e (%): 591 (M+H⁺, 100).

### EXAMPLE 3

### (S)-2-(3,5-Bis-trifluoromethyl-phenyl)-N-[4-(4-fluoro-2-methyl-phenyl)-6-(pyrrolidin-2-ylmethoxy)-pyridin-3-yl]-N-methyl-isobutyramide

A mixture of 0.20 g (0.38 mmol) 2-(3,5-bis-trifluoromethyl-phenyl)-N-[6-chloro-4-(4-fluoro-2-methyl-phenyl)-pyridin-3-yl]-N-methyl-isobutyramide, 0.042 g (0.41 mmol) L-prolinol, 0.003 g (0.009 mmol) cetyltrimethylammonium bromide, 0.01 g (0.02 mmol) bis(tri-tert-butylphosphine)palladium(0), 0.05 ml NaOH 50% and 1.2 ml toluene was degassed by two freeze-thaw cycles. The reaction mixture was heated under argon at 90° C. for 3 days. After cooling to room temperature the mixture was diluted with water and extracted with three portions of dichloromethane. The combined organic layers were dried over sodium sulphate and concentrated in vacuo. Flash column chromatography gave 44 mg (20%) of the title compound as a light yellow solid.

MS m/e (%): 598 (M+H⁺, 100).

### EXAMPLE 4

### 2-(3,5-Bis-trifluoromethyl-phenyl)-N-[4-(4-fluoro-2-methyl-phenyl)-6-(2-hydroxy-ethylsulfanyl)-pyridin-3-yl]-N-methyl-isobutyramide

A mixture of 0.25 g (0.47 mmol) 2-(3,5-bis-trifluoromethyl-phenyl)-N-[6-chloro-4-(4-fluoro-2-methyl-phenyl)-pyridin-3-yl]-N-methyl-isobutyramide, 1.1 g (14 mmol) 2-mercapto-ethanol and 0.20 g (1.4 mmol) potassium carbonate was heated under argon at 140° C. for 3 h. After cooling to room temperature the mixture was diluted with water and extracted with three portions of tert-butyl methyl ether. The combined organic layers were dried over sodium sulphate and concentrated in vacuo. Flash column chromatography gave 0.13 g (50%) of the title compound as a white solid.

MS m/e (%): 575 (M+H⁺, 100).

### EXAMPLE 5

### 2-(3,5-Bis-trifluoromethyl-phenyl)-N-[4-(4-fluoro-2-methyl-phenyl)-[2,3′]bipyridinyl-5-yl]-N-methyl-isobutyramide

To a solution of 0.50 g (3.2 mmol) 3-bromopyridine in 6 ml dry THF were added 1.6 ml (3.2 mmol) of a 2 M solution of isopropyl magnesium chloride in THF at −60° C. The resulting orange solution was kept at −40° C. for 15 min and was consequently allowed to warm to room temperature. After 2 h 4.9 ml (4.9 mmol) of a 1 M solution of zinc chloridein dry THF was added to the orange suspension. This mixture was stirred for another 2 h at room temperature. After addition of a solution of 1.0 g (1.9 mmol) 2-(3,5-bis-trifluoromethyl-phenyl)-N-[6-chloro-4-(4-fluoro-2-methyl-phenyl)-pyridin-3-yl]-N-methyl-isobutyramide and 0.11 g (0.095 mmol) tetrakis(triphenylphosphine)palladium(0) in 6 ml THF the reaction mixture was heated at reflux for 16 h. Cooling to room temperature was followed by quenching with water and an 0.5 M aqueous solution of sodium hydroxide. The mixture was extracted with four portions of dichloromethane. The combined organic extracts were washed with two portions of brine, dried over sodium sulfate and concentrated in vacuo. Flash chromatography gave 0.36 g (33%) of the title compound as an off-white solid.

MS m/e (%): 576 (M+H⁺, 100).

### EXAMPLE 6

### 2-(3,5-Bis-trifluoromethyl-phenyl)-N-[4-(4-fluoro-2-methyl-phenyl)-1′-oxy-[2,3′]bipyridinyl-5-yl]-N-methyl-isobutyramide

To a solution of 70 mg (0.12 mmol) 2-(3,5-bis-trifluoromethyl-phenyl)-N-[4-(4-fluoro-2-methyl-phenyl)-[2,3′]bipyridinyl-5-yl]-N-methyl-isobutyramide in 2 ml dichloromethane were added 33 mg (0.13 mmol) 3-chloroperbenzoic acid at room temperature. After 3 h a portion of silica gel was added to the reaction mixture followed by concentration in vacuo. The residue was transferred to a flash chromatography column. Elution gave 64 mg (89%) of the title compound as a white solid.

MS m/e (%): 592 (M+H⁺, 100).

### EXAMPLE 7

### 2-(3,5-Bis-trifluoromethyl-phenyl)-N-[4-(4-fluoro-2-methyl-phenyl)-6-(3-hydroxymethyl-phenyl)-pyridin-3-yl]-N-methyl-isobutyramide

A mixture of 126 mg (0.236 mmol) 2-(3,5-bis-trifluoromethyl-phenyl)-N-[6-chloro-4-(4-fluoro-2-methyl-phenyl)-pyridin-3-yl]-N-methyl-isobutyramide, 40 mg (0.26 mmol) 3-(hydroxymethyl)phenylboronic acid, 0.5 ml of a 2 M aqueous sodium carbonate solution and 2 ml 1,2-dimethoxyethane was degassed by three freeze-thaw cycles. After addition of 3 mg (0.01 mmol) palladium acetate and 6 mg (0.02 mmol) triphenylphosphine the reaction mixture was stirred under argon at 90° C. for 12 h. After cooling to room temperature the reaction mixture was diluted with 2 M sodium carbonate solution and extracted with three portions of tert-butyl methyl ether. The combined organic layers were washed with brine, dried over sodium sulfate and concentrated in vacuo. Flash chromatography gave 117 mg (82%) of the title compound as a white solid.

MS m/e (%): 605 (M+H⁺, 100).

### EXAMPLE 8

### 2-(3,5-Bis-trifluoromethyl-phenyl)-N-[4-(4-fluoro-2-methyl-phenyl)-5′-hydroxymethyl-[2,3′]bipyridinyl-5-yl]-N-methyl-isobutyramide

### 5-Benzyloxy-nicotinic acid methyl ester

To a solution of 13.5 g (88.2 mmol) 5-hydroxynicotinic acid methyl ester in 220 ml DMF were added in small portions 4.6 g (97 mmol) sodium hydride (55% dispersion in oil) at 0° C. After stirring at this temperature for 1 h a solution of 11 ml (93 mmol) benzyl bromide in 40 ml DMF was added dropwise over a period of 15 min. After completed addition the reaction mixture was allowed to warm to room temperature over night. The mixture was diluted with water and extracted with five portions of tert-butyl methyl ether. The combined organic extracts were washed with two portions of water, dried over sodium sulfate and concentrated in vacuo. Flash column chromatography gave 10.7 g (50%) of the title compound as a light yellow solid.

### (5-Benzyloxy-pyridin-3-yl) -methanol

To a solution of 12.2 g (50.0 mmol) 5-benzyloxy-nicotinic acid methyl ester in 250 ml toluene was added a solution of 0.69 g (30 mmol) lithium borohydride in 30 ml THF at room temperature. The mixture was stirred at 100° C. for 5 h. After cooling to 0° C., 10 ml of water and 60 ml of a 1 M aqueous hydrochloric acid solution were added dropwise. Basification with 80 ml of a 2 M aqueous solution of sodium hydroxide and dilution with 200 ml of water was followed by extraction with four portions of tert-butyl methyl ether. The combined organic extracts were washed with water and brine, dried over sodium sulfate and concentrated in vacuo. Flash column chromatography gave 6.4g (59%) of the title compound as an off-white solid.

### 3-Benzyloxy-5-(tert-butyl-dimethyl-silanyloxymethyl)-pyridine

To a solution of 0.75 g (3.5 mmol) (5-benzyloxy-pyridin-3-yl)-methanol and 0.52 g (7.7 mmol) imidazole in 12 ml DMF were added 0.58 g (3.8 mmol) tert-butyldimethylsilyl chloride at room temperature. The mixture was stirred for 3 days. Dilution with a 0.2 M aqueous solution of sodium hydroxide was followed by extraction with three portions of tert-butyl methyl ether. The combined organic extracts were washed with water and brine, dried over sodium sulfate and concentrated in vacuo. Flash column chromatography gave 1.1 g (98%) of the title compound as a light yellow oil.

MS m/e (%): 330 (M+H⁺, 100).

### 5-(tert-Butyl-dimethyl-silanyloxymethyl)-pyridin-3-ol

A mixture of 1.1 g (3.4 mmol) 3-benzyloxy-5-(tert-butyl-dimethyl-silanyloxymethyl)-pyridine and 0.36 g palladium on charcoal (10%) in 17 ml methanol was stirred under an atmosphere of hydrogen at room temperature for 2 h. The mixture was filtered over Decalite and the filtrate was concentrated in vacuo to give 0.78 g (96%) of the crude title compound as a light yellow solid.

MS m/e (%): 240 (M+H⁺, 100).

### Trifluoro-methanesulfonic acid 5-(tert-butyl-dimethyl-silanyloxymethyl)-pyridin-3-yl ester

To a solution of 0.78 g (3.3 mmol) 5-(tert-butyl-dimethyl-silanyloxymethyl)-pyridin-3-ol and 0.66 g (6.5 mmol) triethylamine in 25 ml dichloromethane was added dropwise over a period of 20 min a solution of 1.1 g (3.9 mmol) trifluoromethanesulfonic anhydride in 8 ml dichloromethane at 0° C. After 20 min the reaction mixture was diluted with water and extracted with two portions of dichloromethane. The combined organic extracts were washed with a saturated solution of sodium hydrogencarbonate, dried over sodium sulfate and concentrated in vacuo. Flash column chromatography gave 0.57 g (47%) of the title compound as a light yellow amorphous resin.

MS m/e (%): 372 (M+H⁺, 4).

### 2-(3,5-Bis-trifluoromethyl-phenyl)-N-[4-(4-fluoro-2-methyl-phenyl)-5′-hydroxymethyl-[2,3′]bipyridinyl-5-yl]-N-methyl-isobutyramide

A mixture of 0.15 g (0.41 mmol) trifluoro-methanesulfonic acid 5-(tert-butyl-dimethyl-silanyloxymethyl)-pyridin-3-yl ester, 0.12 g (0.45 mmol) bis(pinacolato)diboron and 0.12 g (1.2 mmol) potassium acetate in 4 ml N,N-dimethylformamide was deoxygenated by three freeze-thaw cycles. After addition of 46 mg (0.056 mmol) dichloro(1,1′-bis(diphenylphosphino)ferrocene)palladium(II) dichloromethane adduct the reaction mixture was stirred at 80° C. over night. Cooling to room temperature was followed by addition of 2 ml of a 2 M aqueous solution of sodium carbonate, 0.20 g (0.38 mmol) 2-(3,5-bis-trifluoromethyl-phenyl)-N-[6-chloro-4-(4-fluoro-2-methyl-phenyl)-pyridin-3-yl]-N-methyl-isobutyramide and 23 mg (0.028 mmol) dichloro(1,1′-bis(diphenylphosphino)ferrocene)palladium(II) dichloromethane adduct. The reaction mixture was heated at 80° C. over night. After cooling to room temperature the reaction mixture was diluted with a 0.1 M aqueous solution of sodium hydroxide and extracted with three portions of tert-butyl methyl ether. The combined organic extracts were washed with water and brine, dried with sodium sulfate, filtered and concentrated. The residue was dissolved in 4 ml of a mixture of methanol and concentrated aqueous hydrochloric acid (95:5) and stirred at room temperature for 90 min. The mixture was diluted with excess 1 M sodium hydroxide solution and extracted with three portions of tert-butyl methyl ether. The combined organic extracts were dried over sodium sulfate and concentrated. Flash column chromatography gave 32 mg (14%) of the title compound as a light brown solid.

MS m/e (%): 606 (M+H⁺, 100).

### EXAMPLE 9

### 2-(3,5-Bis-trifluoromethyl-phenyl)-N-[4-(4-fluoro-2-methyl-phenyl)-2′-hydroxymethyl-[2,4′]bipyridinyl-5-yl]-N-methyl-isobutyramide

### 2-(3,5-Bis-trifluoromethyl-phenyl)-N-[4-(4-fluoro-2-methyl-phenyl)-2′-methyl-[2,4′]bipyridinyl-5-yl]-N-methyl-isobutyramide

A mixture of 90 mg (0.41 mmol) 4-iodo-2-methyl-pyridine, 0.12 g (0.45 mmol) bis(pinacolato)diboron and 0.12 g (1.2 mmol) potassium acetate in 4 ml N,N-dimethylformamide was deoxygenated by three freeze-thaw cycles. After addition of 46 mg (0.056 mmol) dichioro(1,1′-bis(diphenylphosphino)ferrocene)palladium(II) dichloromethane adduct the reaction mixture was stirred at 80° C. over night. Cooling to room temperature was followed by addition of 2 ml of a 2 M aqueous solution of sodium carbonate, 0.20 g (0.38 mmol) 2-(3,5-bis-trifluoromethyl-phenyl)-N-[6-chloro-4-(4-fluoro-2-methyl-phenyl)-pyridin-3-yl]-N-methyl-isobutyramide and 23 mg (0.028 mmol) dichloro(1,1′-bis(diphenylphosphino)ferrocene)palladium(II) dichloromethane adduct. The reaction mixture was heated at 80° C. over night. After cooling to room temperature the reaction mixture was diluted with a 0.1 M aqueous solution of sodium hydroxide and extracted with three portions of tert-butyl methyl ether. The combined organic extracts were washed with water and brine, dried with sodium sulfate, filtered and concentrated. Flash column chromatography gave 28 mg (13%) of the title compound as a light yellow solid.

### 2-(3,5-Bis-trifluoromethyl-phenyl)-N-[4-(4-fluoro-2-methyl-phenyl)-2′-methyl-1′-oxy-[2,4′]bipyridinyl-5-yl]-N-methyl-isobutyramide

The title compound was obtained in 97% yield according to the procedure described above for the preparation of 2-(3,5-bis-trifluoromethyl-phenyl)-N-[4-(4-fluoro-2-methyl-phenyl)-1′-oxy-[2,3′]bipyridinyl-5-yl]-N-methyl-isobutyramide (example 6) using 2-(3,5-bis-trifluoromethyl-phenyl)-N-[4-(4-fluoro-2-methyl-phenyl)-2′-methyl-[2,4′]bipyridinyl-5-yl]-N-methyl-isobutyramide instead of 2-(3,5-bis-trifluoromethyl-phenyl)-N-[4-(4-fluoro-2-methyl-phenyl)-[2,3′]bipyridinyl-5-yl]-N-methyl-isobutyramide.

MS m/e (%): 606 (M+H⁺, 100).

### 2-(3,5-Bis-trifluoromethyl-phenyl)-N-[4-(4-fluoro-2-methyl-phenyl)-2′-hydroxymethyl-[2,4′]bipyridinyl-5-yl]-N-methyl-isobutyramide

A solution of 49 mg (0.081 mmol) 2-(3,5-bis-trifluoromethyl-phenyl)-N-[4-(4-fluoro-2-methyl-phenyl)-2′-methyl-1′-oxy-[2,4′]bipyridinyl-5-yl]-N-methyl-isobutyramide and 51 mg (0.24 mmol) trifluoroacetic anhydride was stirred at room temperature over night. Another 34 mg (0.16 mmol) trifluoroacetic anhydride were added and stirring was continued for 20 h. After addition of methanol the reaction mixture was concentrated in vacuo. Flash column chromatography gave 31 mg (63%) of the title compound as a white solid.

MS m/e (%): 606 (M+H⁺, 100).

### EXAMPLE 10

### 2-(3,5-Bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide

### 5-[2-(3,5-Bis-trifluoromethyl-phenyl)-2-methyl-propionyl]-methyl-amino]-4-(4-fluoro-2-methyl-phenyl)-3′,6′-dihydro-2′H-[2,4′]bipyridinyl-1′-carboxylic acid tert-butyl ester

The title compound was obtained as a white solid in 47% yield according to the procedure described above for the preparation of 2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide (example 7) using 4-(4,4,5,5-tetramethyl-[1,3,2]dioxaborolan-2-yl)-3,6-dihydro-2H-pyridine-1-carboxylic acid tert-butyl ester instead of 3-(hydroxymethyl)phenylboronic acid. 4-(4,4,5,5-Tetramethyl-[1,3,2]dioxaborolan-2-yl)-3,6-dihydro-2H-pyridine-1-carboxylic acid tert-butyl ester was prepared as described by P. Eastwood, Tetrahedron Lett. 2000, 41, 3705.

MS m/e (%): 680 (M+H⁺, 100).

### 2-(3,5-Bis-trifluoromethyl-phenyl)-N-[4-(4-fluoro-2-methyl-phenyl)-1′,2′,3′,6′-tetrahydro-[2,4′]bipyridinyl-5-yl]-N-methyl-isobutyramide

As solution of 0.12 g (0.18 mmol) 5-{[2-(3,5-bis-trifluoromethyl-phenyl)-2-methyl-propionyl]-methyl-amino}-4-(4-fluoro-2-methyl-phenyl)-3′,6′-dihydro-2′H-[2,4′]bipyridinyl-1′-carboxylic acid tert-butyl ester and 0.50 ml (6.5 mmol) trifluoroacetic acid in 1.5 ml dichloromethane was stirred at room temperature for 15 min. The mixture was basified by the addition of 2 M aqueous sodium hydroxide solution and extracted with three portions of dichloromethane. The combined organic extracts were dried over sodium sulfate and concentrated in vacuo to give 0.10 g (99%) of the crude title compound as an off-white solid.

MS m/e (%): 580 (M+H⁺, 100).

### 2-(3,5-Bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide

To a solution of 0.10 g (0.17 mmol) 2-(3,5-bis-trifluoromethyl-phenyl)-N-[4-(4-fluoro-2-methyl-phenyl)-1′,2′,3′,6′-tetrahydro-[2,4′]bipyridinyl-5-yl]-N-methyl-isobutyramide in 2 ml dichloromethane were added 21 mg (0.21 mmol) triethylamine and 21 mg (0.18 mmol) methanesulfonyl chloride at 0° C. After completed addition the reaction mixture was allowed to warm to room temperature during 30 min. Dilution with water was followed by extraction with three portions dichloromethane. The combined organic layers were dried over sodium sulfate, concentrated and purified by flash chromatography to give 77 mg (68%) of the title compound as a white solid.

MS m/e (%): 658 (M+H⁺, 100).

### EXAMPLE 11

### 2-(3,5-Bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide

### 2-(3,5-Bis-trifluoromethyl-phenyl)-N-[4-(4-fluoro-2-methyl-phenyl)-[2,4′]bipyridinyl-5-yl]-N-methyl-isobutyramide

The title compound was obtained as an off-white solid in 70% yield according to the procedure described above for the preparation of 2-(3,5-bis-trifluoromethyl-phenyl)-N-[4-(4-fluoro-2-methyl-phenyl)-[2,3′]bipyridinyl-5-yl]-N-methyl-isobutyramide (example 5) using 4-iodopyridine instead of 3-bromopyridine.

MS m/e (%): 576 (M+H⁺, 100).

### 2-(3,5-Bis-trifluoromethyl-phenyl)-N-[4-(4-fluoro-2-methyl-phenyl)-1′,2′,3′,4′,5′,6′-hexahydro-[2,4′]bipyridinyl-5-yl]-N-methyl-isobutyramide

A solution of 0.20 g (0.35 mmol) 2-(3,5-bis-trifluoromethyl-phenyl)-N-[4-(4-fluoro-2-methyl-phenyl)-[2,4′]bipyridinyl-5-yl]-N-methyl-isobutyramide and 0.020 ml (0.35 mmol) concentrated sulfuric acid in 4 ml methanol was degassed by three freeze-thaw cycles. After addition of 39 mg (0.17 mmol) platinum(IV)oxide under argon the reaction mixture was stirred under an atmosphere of hydrogen at room temperature for 16 h. The mixture was concentrated in vacuo. The residue was partitioned between 1 M aqueous sodium hydroxide solution and dichloromethane and extracted with three portions of dichloromethane. The combined organic extracts were dried over sodium sulfate and concentrated to give 0.19 g (94%) of the crude title compound as a brown solid.

MS m/e (%): 582 (M+H⁺, 100).

### 2-(3,5-Bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide

The title compound was obtained as an off-white solid in 79% yield according to the procedure described above for the preparation of 2-(3,5-bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide (example 10 c)) using 2-(3,5-bis-trifluoromethyl-phenyl)-N-[4-(4-fluoro-2-methyl-phenyl)-1′,2′,3′,4′,5′,6′-hexahydro-[2,4′]bipyridinyl-5-yl]-N-methyl-isobutyramide instead of 2-(3,5-bis-trifluoromethyl-phenyl)-N-[4-(4-fluoro-2-methyl-phenyl)-1′,2′,3′,6′-tetrahydro-[2,4′]bipyridinyl-5-yl]-N-methyl-isobutyramide.

MS m/e (%): 660 (M+H⁺, 100).

### EXAMPLE 12

### (RS)-2-(3,5-Bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide

The title compound was obtained as an off-white solid in comparable yield according to the procedures described above for the preparation of 2-(3,5-bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide (example 11, steps b) and c)) using 2-(3,5-bis-trifluoromethyl-phenyl)-N-[4-(4-fluoro-2-methyl-phenyl)-[2,3′]bipyridinyl-5-yl]-N-methyl-isobutyramide (example 5) instead of 2-(3,5-bis-trifluoromethyl-phenyl)-N-[4-(4-fluoro-2-methyl-phenyl)-[2,4′]bipyridinyl-5-yl]-N-methyl-isobutyramide in step b).

MS m/e (%): 660 (M+H⁺, 100).

### EXAMPLE 13

### (RS)-N-[1′-Acetyl-4-(4-fluoro-2-methyl-phenyl)-1′,2′,3′,4′,5′,6′-hexahydro-[2,3′]bipyridinyl-5-yl]-2-(3,5-bis-trifluoromethyl-phenyl)-N-methyl-isobutyramide

The title compound was obtained as an off-white solid in comparable yield according to the procedures described above for the preparation of (RS)-2-(3,5-bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide (example 12) using acetic anhydride instead of methanesulfonyl chloride in the last step.

MS m/e (%): 624 (M+H⁺, 100).

### EXAMPLE 14

### 2-(3,5-Bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide

### 2-(3,6-Dihydro-2H-thiopyran-4-yl)-4,4,5,5-tetramethyl-[1,3,2]dioxaborolane

To a solution of 1.3 ml (9.0 mmol) diisoproylamine in 5 ml dry THF were added 5.7 ml (9.0 mmol) of a 1.6 M solution of n-butyllithium in hexanes at −78° C. After completed addition the mixture was allowed to warm to 0° C. To this solution was added dropwise a solution of 1.0 g (8.6 mmol) tetrahydro-4H-thiopyranone in 5 ml THF at −78° C. After 30 min a solution of 3.1 g (8.8 mmol) N-phenyl-bis(trifluoromethanesulfonimid) in 8 ml THF was added dropwise. The reaction mixture was allowed to warm to room temperature and stirred at this temperature for 4 h. The solvent was evaporated in vacuo and the residue was purified by flash column chromatography to give 2.1 g (98%) trifluoro-methanesulfonic acid 3,6-dihydro-2H-thiopyran-4-yl ester.

A mixture of 2.0 g (8.1 mmol) trifluoro-methanesulfonic acid 3,6-dihydro-2H-thiopyran-4-yl ester, 2.3 g (8.9 mmol) bis(pinacolato)diboron, 0.18 g (0.24 mmol) dichloro(1,1′-bis(diphenylphosphino)ferrocene)palladium(II) dichloromethane, 0.13 g (0.24 mmol) 1,1′-bis(diphenylphosphino)ferrocene and 2.4 g (24 mmol) potassium acetate in 20 ml dioxane was stirred at 80° C. for 16 h. After cooling to room temperature the reaction mixture was diluted with water and brine (1:1) and extracted with three portions of tert-butyl methyl ether. The combined organic extracts were washed with brine, dried over sodium sulfate and concentrated in vacuo. Flash column chromatography gave 0.97 g (53%) of the title compound as an orange resin.

### 2-(3,5-Bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide

The title compound was obtained as a light yellow solid in 73% yield according to the procedure described above for the preparation of 2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide (example 7) using 2-(3,6-dihydro-2H-thiopyran-4-yl)-4,4,5,5-tetramethyl-[1,3,2]dioxaborolane instead of 3-(hydroxymethyl)phenylboronic acid.

MS m/e (%): 597 (M+H⁺, 100).

### EXAMPLE 15

### 2-(3,5-Bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide

To a solution of 0.24 g (0.40 mmol) 2-(3,5-bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide in 4 ml dichloromethane were added 0.21 g (0.84 mmol) 3-chloroperbenzoic acid at 0° C. After 3 h the reaction mixture was diluted with a 0.15 M aqueous solution of sodium hydroxide and extracted with three portions of dichloromethane. The combined organic extracts were dried over sodium sulfate and concentrated in vacuo. Flash column chromatography gave 0.23 g (92%) of the title compound as an off-white solid.

MS m/e (%): 629 (M+H⁺, 100).

### EXAMPLE 16

### 2-(3,5-Bis-trifluoromethyl-phenyl)-N-[6-(1,1-dioxo-hexahydro-1λ⁶-thiopyran-4-yl)-4-(4-fluoro-2-methyl-phenyl)-pyridin-3-yl]-N-methyl-isobutyramide

A solution of 0.10 g (0.16 mmol) 2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide and 1 drop of perchloric acid (70%) in 3 ml ethyl acetate was degassed by three freeze-thaw cycles. After addition of 11 mg (0.048 mmol) platinum(IV)oxide under argon the reaction mixture was stirred under an atmosphere of hydrogen at room temperature for 6 h. The mixture was filtered over Decalite and the filtrate was concentrated in vacuo. Flash column chromatography gave 32 mg (32%) of the title compound as a white solid.

MS m/e (%): 631 (M+H⁺, 100).

### EXAMPLE A

Tablets of the following composition are manufactured in the usual manner:

| | mg/tablet |
|---|---|
| Active substance | 5 |
| Lactose | 45 |
| Corn starch | 15 |
| Microcrystalline cellulose | 34 |
| Magnesium stearate | 1 |
| Tablet weight | 100 |

### EXAMPLE B

Capsules of the following composition are manufactured:

| | mg/capsule |
|---|---|
| Active substance | 10 |
| Lactose | 155 |
| Corn starch | 30 |
| Talc | 5 |
| Capsule fill weight | 200 |

The active substance, lactose and corn starch are firstly mixed in a mixer and then in a comminuting machine. The mixture is returned to the mixer, the talc is added thereto and mixed thoroughly. The mixture is filled by machine into hard gelatin capsules.

### EXAMPLE C

Suppositories of the following composition are manufactured:

| | mg/supp. |
|---|---|
| Active substance | 15 |
| Suppository mass | 1285 |
| Total | 1300 |

The suppository mass is melted in a glass or steel vessel, mixed thoroughly and cooled to 45° C.

Thereupon, the finely powdered active substance is added thereto and stirred until it has dispersed completely. The mixture is poured into suppository moulds of suitable size, left to cool, the suppositories are then removed from the moulds and packed individually in wax paper or metal foil.

1. A method of treating schizophrenia comprising administering to an individual a therapeutically effective amount of a compound of formula I 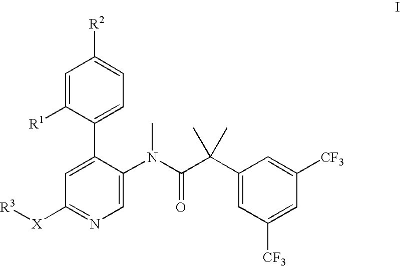
wherein
R¹ is lower alkyl or halogen;
R² is hydrogen or halogen;
R³ —(CHR′)ₙOH,
phenyl optionally substituted by —(CHR′)ₙOH,
a saturated, partially saturated or aromatic 5-or 6-membered heterocyclic ring with one heteroatom or heterogroup, selected from the group consisting of —N(R⁴)—, —N═, 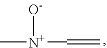
—S— or —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
or a pharmaceutically active acid-addition salt thereof:

2. The method of claim 1, wherein X is —O— or —CH₂O—.

3. The method of claim 2, wherein the compound of formula I is selected from the group consisting of
2-(3,5-bis-trifluoromethyl-phenyl)-N-[4-(2-chloro-phenyl)-6-(2-hydroxy-ethoxy)-pyridin-3-yl]-N-methyl-isobutyramide,
2-(3,5-bis-trifluoromethyl-phenyl)-N-[4-(2-chloro-phenyl)-6-(2-hydroxy-1-hydroxymethyl-ethoxy)-pyridin-3-yl]-N-methyl-isobutyramide and
(S)-2-(3,5-bis-trifluoromethyl-phenyl)-N-[4-(4-fluoro-2-methyl-phenyl)-6-(pyrrolidin-2-ylmethoxy)-pyridin-3-yl]-N-methyl-isobutyramide.

4. The method of claim 1, wherein X is —S—.

5. The method of claim 4, wherein the compound is
2-(3,5-bis-trifluoromethyl-phenyl)-N-[4-(4-fluoro-2-methyl-phenyl)-6-(2-hydroxy-ethylsulfanyl)-pyridin-3-yl]-N-methyl-isobutyramide.

6. The method of claim 1, wherein X is a bond and R³ is a saturated, partial saturated or aromatic 5-or 6-membered heterocyclic ring with one heteroatom or heterogroup, selected from the group consisting of —N(R⁴)—, —N═, 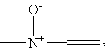
—S— or —S(O)₂, and which rings are optionally substituted by —(CHR′)ₙOH.

7. The method of claim 6, wherein the compound of formula I is selected from the group consisting of
2-(3,5-bis-trifluoromethyl-phenyl)-N-[4-(4-fluoro-2-methyl-phenyl)-[2,3′]bipyridinyl-5-yl]-N-methyl-isobutyramide,
2-(3,5-bis-trifluoromethyl-phenyl)-N-[4-(4-fluoro-2-methyl-phenyl)-1′-oxy-[2,3′]bipyridinyl-5-yl]-N-methyl-isobutyramide,
2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide,
2-(3,5-bis-trifluoromethyl-phenyl)-N-[4-(4-fluoro-2-methyl-phenyl)-5′-hydroxymethyl-[2,3′]bipyridinyl-5-yl]-N-methyl-isobutyramide,
2-(3,5-bis-trifluoromethyl-phenyl)-N-[4-(4-fluoro-2-methyl-phenyl)-2′-hydroxymethyl-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide,
(RS)-2-(3,5-bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide.
(RS)-N-[1′-acetyl-4-(4-fluoro-2-methyl-phenyl)-1′,2′,3′,4′,5′,6′-hexahydro-[2,3′]bipyridinyl-5-yl]-2-(3,5-bis-trifluoromethyl-phenyl)-N-methyl-isobutyramide,
2-(3,5-bis-trifluoromethyl-phenyl)-N-[6-(3,6-dihydro-2H-thiopyran-4-yl)-4-(4-fluoro-2-methyl-phenyl)-pyridin-3-yl]-N-methyl-isobutyramide,
2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide and
2-(3,5-bis-trifluoromethyl-phenyl)-N-[6-(1,1-dioxo-hexahydro-1λ⁶-thiopyran-4-yl)-4-(4-fluoro-2-methyl-phenyl)-pyridin-3-yl]-N-methyl-isobutyramide.

8. A compound of formula I 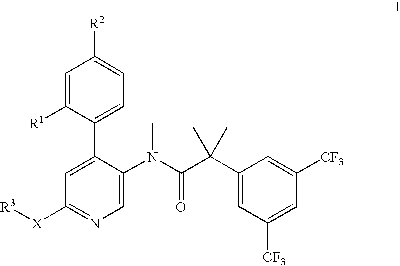
wherein
R¹ is lower alkyl or halogen;
R² is hydrogen or halogen;
R³ —(CHR′)ₙOH,
phenyl optionally substituted by —(CHR′)ₙOH,
a saturated, partially saturated or aromatic 5-or 6-membered heterocyclic ring with one heteroatom or heterogroup, selected from the group consisting of —N(R⁴)—, —N═, 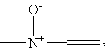
—S— or —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
or a pharmaceutically active acid-addition salt thereof selected from the group consisting of
2-(3,5-bis-trifluoromethyl-phenyl)-N-[4-(2-chloro-phenyl)-6-(2-hydroxy-ethoxy)-pyridin-3-yl]-N-methyl-isobutyramide,
2-(3,5-bis-trifluoromethyl-phenyl)-N-[4-(2-chloro-phenyl)-6-(2-hydroxy-1-hydroxymethyl-ethoxy)-pyridin-3-yl]-N-methyl-isobutyramide,
(S)-2-(3,5-bis-trifluoromethyl-phenyl)-N-[4-(4-fluoro-2-methyl-phenyl)-6)-pyrrolidin-2-ylmethoxy)-pyridin-3-yl]-N-methyl-isobutyramide.
2-(3,5-bis-trifluoromethyl-phenyl)-N-[4-(4-fluoro-2-methyl-phenyl)-6-(2-hydroxy-ethylsulfanyl)-pyridin-3-yl]-N-methyl-isobutyramide.
2-(3,5-bis-trifluoromethyl-phenyl)-N-[4-(4-fluoro-2-methyl-phenyl)-[2,3′]bipyridinyl-5-yl]-N-methyl-isobutyramide,
2-(3,5-bis-trifluoromethyl-phenyl)-N-[4-(4-fluoro-2-methyl-phenyl)-1′-oxy-[2,3′]bipyridinyl-5-yl]-N-methyl-isobutyramide,
2-(3,5-bis-trifluoromethyl-phenyl)-N-[4-(4-fluoro-2-methyl-phenyl)-6-(3-hydroxymethyl-phenyl)-pyridin-3-yl]-N-methyl-isobutyramide, and
2-(3,5-bis-trifluoromethyl-phenyl)-N-[4-(4-fluoro-2-methyl-phenyl)-5′-hydroxymethyl-[2,3′]bipyridinyl-5-yl]-N-methyl-isobutyramide.

9. A compound of formula I 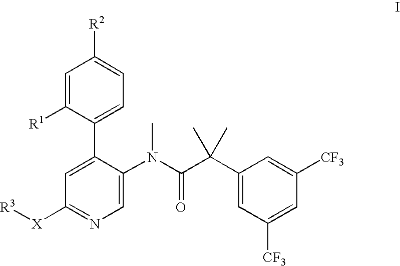
wherein
R¹ is lower alkyl or halogen;
R² is hydrogen or halogen;
R³ —(CHR′)ₙOH,
phenyl optionally substituted by —(CHR′)ₙOH,
a saturated, partially saturated or aromatic 5-or 6-membered heterocyclic ring with one heteroatom or heterogroup, selected from the group consisting of —N(R⁴)—, —N═, 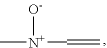
—S— or —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
or a pharmaceutically active acid-addition salt thereof selected from the group consisting of
2-(3,5-bis-trifluoromethyl-phenyl)-N-[4-(4-fluoro-2-methyl-phenyl)-2′-hydroxymethyl-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,6′-tetrahydro-[2,4′]bipyridinyl-5-yl]-N-methyl-isobutyramide,
2-(3,5-bis-trifluoromethyl-phenyl)-N-[4-(4-fluoro-2-methyl-phenyl)-1′-methanesulfonyl-1′,2′,3′,4′,5′,6′-hexahydro-[2,4′]bipyridinyl-5-yl]-N-methyl-isobutyramide,
(RS)-2-(3,5-bis-trifluoromethyl-phenyl)-N-[4-(4-fluoro-2-methyl-phenyl)-1′-methanesulfonyl-1′,2′,3′,4′,5′,6′-hexahydro-[2,3′]bipyridinyl-5-yl]-N-methyl-isobutyramide,
(RS)-N-[1′-acetyl-4)-4-fluoro-2-methyl-phenyl)-1′,2′,3′,4′,5′,6′-hexahydro-[2,3′]bipyridinyl-5-yl]-2-(3,5-bis-trifluoromethyl-phenyl)-N-methyl-isobutyramide,
2-(3,5-bis-trifluoromethyl-phenyl)-N-[6-(3,6-dihydro-2H-thiopyran-4-yl)-4)-4-fluoro-2-methyl-phenyl)-pyridin-3-yl]-N-methyl-isobutyramide,
2-(3,5-bis-trifluoromethyl-phenyl)-N-[6-(1,1-dioxo-1,2,3,6-tetrahydro-1λ⁶-thiopyran-4-yl)-4-(4-fluoro-2-methyl-phenyl)-pyridin-3-yl]-N-methyl-isobutyramide and
2-(3,5-bis-trifluoromethyl-phenyl)-N-[6-(1,1-dioxo-hexahydro-1λ⁶-thiopyran-4-yl)-4-(4-fluoro-2-methyl-phenyl)-pyridin-3-yl]-N-methyl-isobutyramide.

10. A process for preparing a compound of formula I 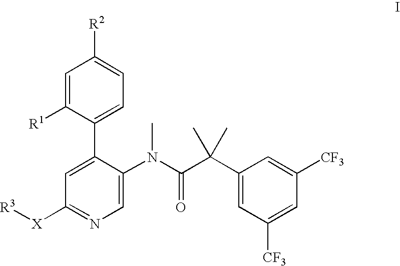
wherein
R¹ is lower alkyl or halogen;
R² is hydrogen or halogen;
R³ —(CHR′)ₙOH,
phenyl optionally substituted by —(CHR′)ₙOH,
a saturated, partially saturated or aromatic 5-or 6-membered heterocyclic ring with one heteroatom or heterogroup, selected from the group consisting of —N(R⁴)—, —N═, 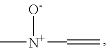
—S— or —S(O)₂, and which rings are optionally substituted by —(CHR′)ₙOH;
R′ is independently from “n” hydrogen or —(CH₂)ₙOH;
R⁴ is hydrogen, —S(O₂)-lower alkyl or —C(O)-lower alkyl;
X is —O—, —CH₂O—, —S— or a bond; and
n is 1 or 2;
selected from the following group of processes
a) reacting a compound of formula 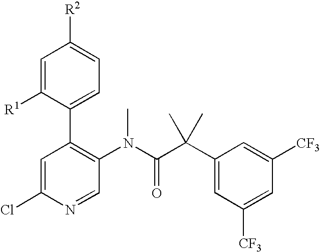
Intermediates 5A-5B with a compound of formula
R³OH
to produce a compound of formula 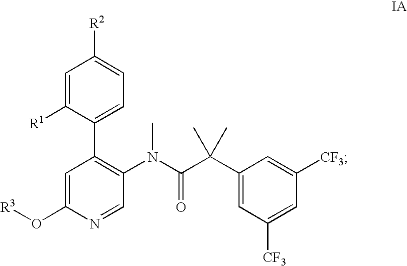
b) reacting a compound of formula 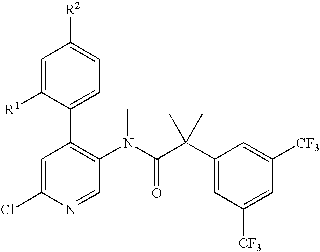
Intermediates 5A-5B with a compound of formula
R³SH
to produce a compound of formula 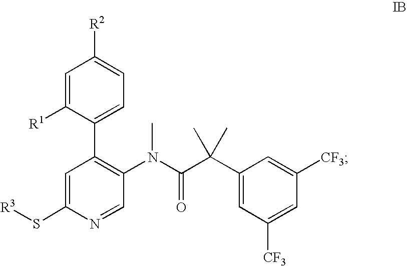
c) reacting a compound of formula 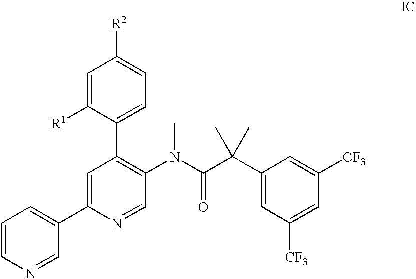
with 3-chloroperbenzoic acid
to produce a compound of formula 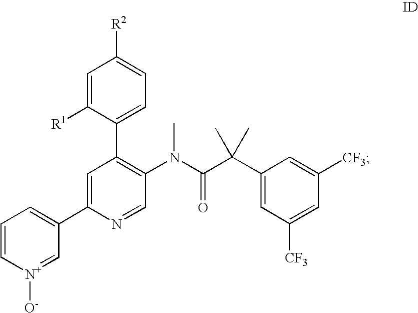
d) reacting a compound of formula 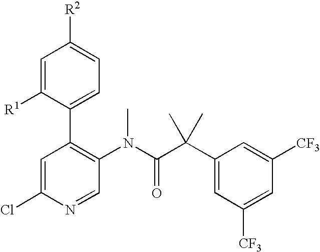
Intermediates 5A-5B with a compound of formula 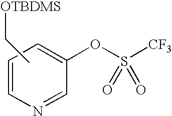
to produce a compound of formula 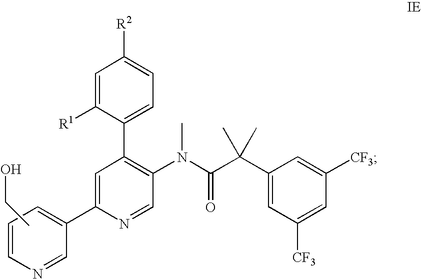
e) reacting a compound of formula 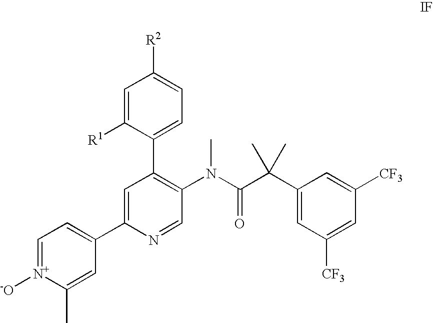
with a compound of formula
(CF₃CO)₂O
to produce a compound of formula 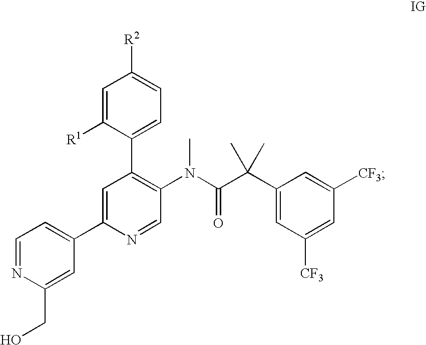
f) reacting a compound of formula 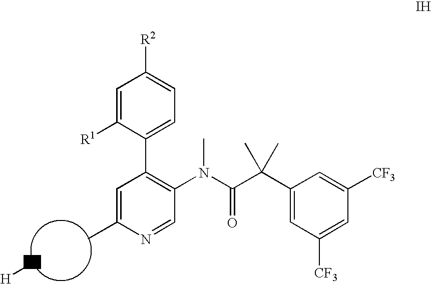
with a compound of formula
CH₃SO₂Cl
to produce a compound of formula 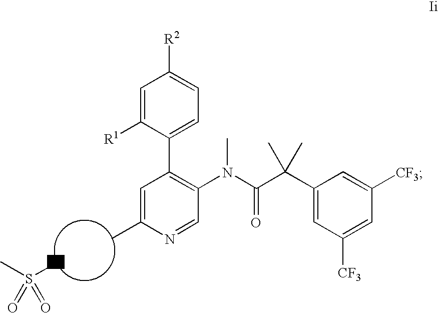
g) reacting a compound of formula 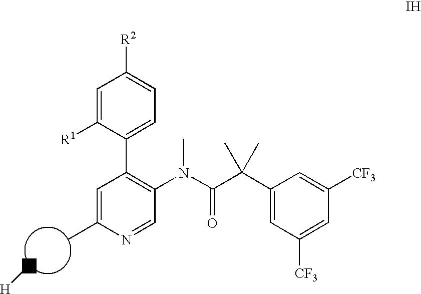
with a compound of formula
(CH₃CO)₂O
to produce a compound of formula 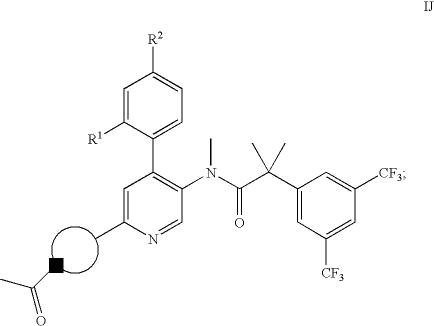
h) reacting a compound of formula 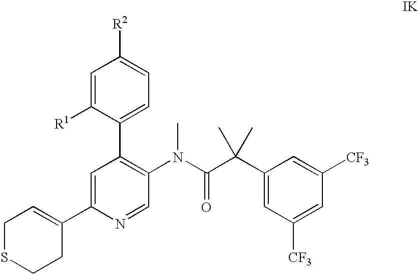
with the compound 3-chloroperbenzoic acid
to produce a compound of formula 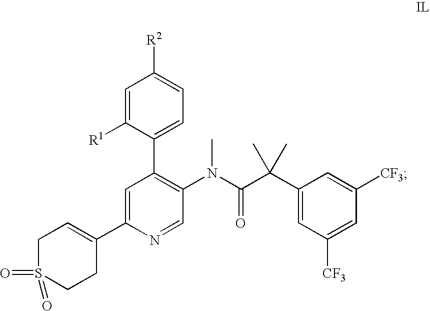
and
i) hydrogenating a compound of formula 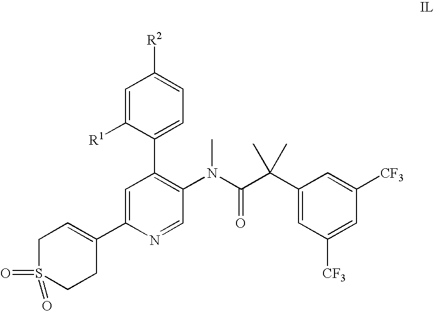
to a compound of formula [chemical structure]
